(19) United States
(12) Patent Application Publication
(10) Pub. No.: US 2008/0139534 A1
(43) Pub. Date: Jun. 12, 2008

(54) Bisbenzamidines and bisbenzamidoximes for the treatment of human African trypanosomiasis

(75) Inventors: Tien L. HUANG, New Orleans, LA (US); Jean Jacques VANDEN EYNDE, Mons (BE); Annie MAYENCE, Mons (BE); Cyrus BACCHI, East Northport, NY (US); Isaac O. DONKOR, Lakeland, TN (US); Nageswara R. KODE, Kenner, LA (US)

Correspondence Address: ADAMS AND REESE LLP, 4400 ONE HOUSTON CENTER, 1221 MCKINNEY, HOUSTON, TX, 77010, US

(73) Assignee: XAVIER UNIVERSITY OF LOUISIANA, New Orleans, LA (US)

(21) Appl. No.: 11/952,455

(22) Filed: Dec. 7, 2007

Related U.S. Application Data
(60) Provisional application No. 60/873,344, filed on Dec. 7, 2006.

(51) Int. Cl. A61K 31/551; A61K 31/167; A61K 31/495; A61P 33/00; C07D 403/14; C07D 413/14; C07D 413/02; C07D 295/155; A61K 31/496; A61K 31/506

(52) U.S. Cl. 514/218; 514/252.14; 514/254.03; 514/254.06; 514/255.03; 514/616; 540/575; 544/296; 544/367; 544/370; 544/392; 548/131; 564/152

(57) ABSTRACT

Disclosed are bisbenzamidine and bisbenzamidoxime compounds useful for treatment of treatment of trypanosomiasis. The compounds disclosed are useful for treating mammals infected with parasitic hemoflagellates, in particular Trypanosoma brucei gambiense and Trypanosoma brucei rhodesiense.

### CROSS-REFERENCE TO RELATED APPLICATIONS

This Non-Provisional Patent Application, filed under 35 U.S.C. §111(a), claims the benefit under 35 U.S.C. §119(e)(1) of U.S. Provisional Patent Application No. 60/873,344, filed under 35 U.S.C. §111(b) on Dec. 07, 2006, and which is hereby incorporated by reference in its entirety.

### STATEMENT REGARDING FEDERALLY SPONSORED RESEARCH OR DEVELOPMENT

The invention was made with U.S. Government support under grant number 2S06GM08008 awarded by the National Institutes of Health. The United States Government has certain rights in the invention.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

Not applicable.

### BACKGROUND OF THE INVENTION

1. Field of the Invention

This invention relates to the development of novel bisbenzamidines and bisbenzamidoximes for the treatment of human African trypanosomiasis caused by Trypanosoma brucei gambiense and Trypanosoma brucei rhodesiense.

2. Description of Related Art

Human African trypanosomiasis (HAT) or sleeping sickness is caused by two subspecies of the parasitic hemoflagellate Trypanosoma brucei: Trypanosoma brucei gambiense and Trypanosoma brucei rhodesiense. Infection with sleeping sickness starts with the bite of an infected insect vector, the tsetse fly (Welburn et al., Parasitol. Today, 1999, 15, 399-403). The parasites are entirely extracellular throughout their life cycle. In the early stage, the parasites move from the site of infection to the draining lymphatic vessels and the bloodstream, where they proliferate and later invade the central nervous system (CNS) in the second stage. Cerebral invasion is responsible for the inflammation of the brain and the spinal cord, that results in the characteristic lethargy and eventually, continous sleep. If left untreated, the disease progresses with increasing breakdown of the neurological function, to coma and eventually death. T. b. gambiense is the chronic form, in which the disease proliferates more slowly and can take several years before spreading into the CNS. T. b. rhodesiense is the acute form of the disease, in which the parasites rapidly invades the CNS, causing death within weeks if untreated. It is estimated that there are currently 300,000 to 500,000 cases of HAT in Africa, with 50,000 deaths reported annually (Fairlamb, A. H., Trends in Parasitology, 2003, 19, 488-494).

The chemotherapy of HAT is restricted to four clinically approved drugs, three of which were introduced over 60 years ago. Only eflornithine was registered in 1990 to treat only the late-stage of T b. gambiense infections. The four approved drugs are suramin, pentamidine, melarsoprol and eflornithine. However, the current therapies are far from satisfactory. These drug regimens are plaque with problems such as unacceptable toxicity, poor efficacy, undesirable route of administration, and drug resistance (Fairlamb, A. H., Trends in Parasitology, 2003, 19, 488-494). Suramin and pentamidine are restricted to the treatment of the early-stage of HAT prior to CNS infection. Melarsoprol, an arsenic based drug, is used once the parasites have penetrated the CNS. Eflornithine is not effective against T. b. rhodiense infections. Based on the limitations of the above drugs, it is widely accepted that new drugs with improved biological properties are urgently needed for the chemotherapy of HAT.

Pentamidine, a bisbenzamidine, is approved for the treatment of early-stage HAT, antimony-resistant leishmaniasis and pneumocystis jerovecii pneumonia in immunocompromised patients. The two benzamidine moieties in pentamidine are joined by a central flexible pentamethylene dioxy chain. It has been demonstrated that the nature of the linker in this class of compounds plays an important role in influencing their biological properties (Tao et al., Eur. J. Med. Chem., 1999, 34, 531-538; Donkor et al., J. Med. Chem., 2003, 46, 1041-1048; Vanden Eynde et al., Med. Chem. Res., 2005, 14, 143-157; Cushion et al., Antimicrob. Agent Chemother., 2006, 50, 2337-2343; Huang et al., J. Pharm. Pharmacol., 2006, 58, 1033-1042). The mechanism of action of pentamidine is not well understood. It has been postulated that pentamidine and other aromatic bisbenzamidines are selectively taken up by transporters into trypanosomes, where they bind strongly to DNA in the nucleus and mitochondria (Bray, P. G. et al., Trends in Parasitology, 2003, 19, 232-239). The strong binding to DNA may result in the inhibition of topoisomerases II resulting in the inhibition of synthesis of DNA, RNA, and proteins by the parasites (Shapiro et al., Proc. Natl. Acad. Sci. U.S.A. 1990, 87, 950-954).

### BRIEF SUMMARY OF THE INVENTION

A series of aromatic bisbenzamidines was synthesized, in which the benzamidine groups were linked with various linkers such as piperazinyl, homopiperazinyl, piperidinyl, phenyl, alkanediamides (e.g. pentanediamide, hexanediamide, etc.), aryldiamides (e.g. phenylene, etc.). The terminal amidinium functions were modified with groups such as amidoxime, O-alkylated amidoxime, oxadiazole, thiadiazole, carbamate, carbonate, or carboxylic ester. The trypanocidal activity of these compounds was evaluated in vitro using a drug sensitive (T. b. brucei Lab 110 EATRO) and a drug-resistant (T. b. rhodesiense KETRI 243) trypanosome isolates and in mouse model infections infected with drug-sensitive (T. b. brucei Lab 110 EATRO) and -resistant clinical isolates (T. b. rhodesiense KETRI 2002, KETRI 2538, KETRI 1992). The binding of these compounds to DNA and Poly (dA-dT) was also determined. These compounds were highly effective against trypanosomes in in vitro and in vivo studies. Development of these compounds could provide new and novel treatment choices for HAT.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

A series of novel bisbenzamidines linked by either a rigid (for instance, a piperazinyl or phenylenediamide) or a flexible (for instance, a pentyldiamide) moiety are described. Such compounds may be used for the treatment of parasitic infections, such as sleeping sickness and for instance, human African trypanosomiasis caused by T. b. gambiense or T. b. rhodesiense. In vitro and in vivo evaluation of these compounds using a drug-sensitive (T. b. brucei Lab 100 EATRO) or drug-resistant (T. b. rhodesiense KETRI 243, KETRI 2002, KETRI 2538 and KETRI 1992) clinical isolates indicated that the bisbenzamidines of the present invention functioned as anti-trpanosomal agents.

The Group I and Group II of compounds described by this invention are summarized by the general structural diagram below:

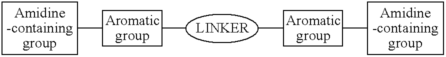

Group I Compounds

Group I compounds comprise two aromatic groups linked together and having at least one aromatic system bearing an amidine or an amidine-containing group. Examples of Group I compounds include those of the general formulae (and salts thereof) of structure 1a and 1b (below):

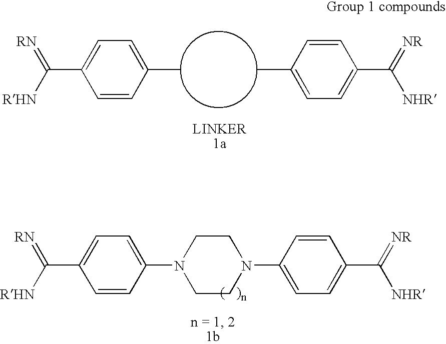

The linker may be any cyclic system of any size and may contain at least one heteroatom. The linker may be aromatic, or, the linker is a 1,4-piperazinediyl group (n=1, 1b) or a 1,4-homopiperazinediyl group (n=2 as shown in structure 1b). The aromatic system is a di-substituted six-membered ring and may contain at least one heteroatom. The aromatic system may also be a fused aryl ring system. The aromatic system is di-substituted, in either the 1,2-; 1,3-; or 1,4- positions.

The R group may be a hydrogen atom or a linear or branched alkyl group, containing from 1 to 20 carbon atoms. R′ may be either a hydrogen atom or a linear or branched alkyl group containing from one to twenty carbon atoms or it may be an aromatic ring. When R is a hydrogen atom, R′ may be a cycloalkyl group containing three to eight carbon atoms. R and R′ may form a cyclic structure that can be fused to another cyclic system. When the substituents R and R′ form a cyclic structure, this structure may be aromatic, and it may contain heteroatoms or unsaturated bonds such as oxadiazoles, thiadiazoles, oxadiazolone, oxadiazolethione, oxathiadiazolone, thiadiazolone and thiadiazolone, or, when the substituent R is hydrogen, R′ may be a hydroxyl, alkyl or aryloxy, alkyl or arylacyl, alkyl or aryl carbonate, alkyl or aryl thiocarbonate group. In addition, R and R′ may contain heteroatoms or unsaturated bonds or other functional groups.

Group II Compounds

Group II compounds comprise two aromatic groups linked together and having at least one di-substituted aromatic system bearing an amidine or an amidine-containing group and also a (de)activating group. These two units are joined by a linking unit of one to twenty carbon atoms. Examples of Group II compounds include those of the general formulae (and salts thereof):

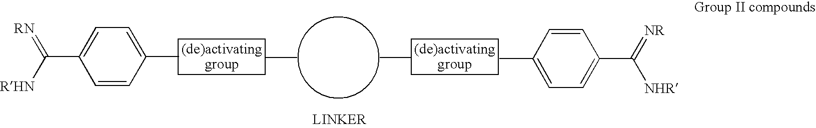

Group II

The linker constitutes a chain of one to twenty carbon atoms, containing saturated and/or unsaturated units, also possibly including a cyclic structure of 1-20 atoms and also possibly containing heteroatoms.

The (de)activating group contains functional groups including but not limited to ether, ester, amide, thioether, thioester, thioamide, amine, or a methylene group.

The aromatic system is di-substituted, six-membered ring and may contain at least one heteroatom. It may also be a fused aryl ring system. The aromatic system is di-substituted in either the 1,2-; 1,3-; or 1,4-positions.

The R group may be a hydrogen atom or a linear or branched alkyl group, containing from 1 to 20 carbon atoms. R′ may be either a hydrogen atom or a linear or branched alkyl group containing from one to twenty carbon atoms or an aromatic ring. When R is a hydrogen atom, R′ may be a cycloalkyl group containing three to eight carbon atoms, or R and R′ may form a cyclic structure that can be fused to another cyclic system. When the substituents R and R′ form a cyclic structure, this structure may be aromatic, and it may contain heteroatoms or unsaturated bonds such as oxadiazoles, thiadiazoles, oxadiazolone, oxadiazolethione, oxathiadiazolone, thiadiazolone and thiadiazolone. When R is hydrogen, R′ may be a hydroxyl, alkyl or aryloxy, alkyl or arylacyl, alkyl or aryl carbonate, alkyl or aryl thiocarbonate group. In addition, R and R′ may contain heteroatoms or unsaturated bonds or other functional groups.

Syntheses

The general strategy used to obtain the derivatives of Group I is shown below (Scheme 1):

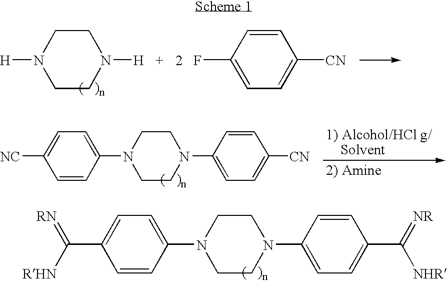

The key step for the preparation of 1,4-diarylpiperazines was a double nucleophilic displacement of fluorine in 4-fluoro derivatives by the nitrogen atoms of piperazine. That reaction, performed in boiling dimethyl formamide, produces the expected tricyclic molecules in good yields, provided the aromatic precursor bears a strong electron-withdrawing group in the para position. Conversion of the bisbenzonitrile derivative into the targeted bisbenzamidines was effected by the Pinner reaction. The bisbenzonitrile can be converted into the bisbenzamidoxime by reacting with hydroxylamine.

The general strategy used to obtain the derivatives of Group II is shown below (Scheme 2):

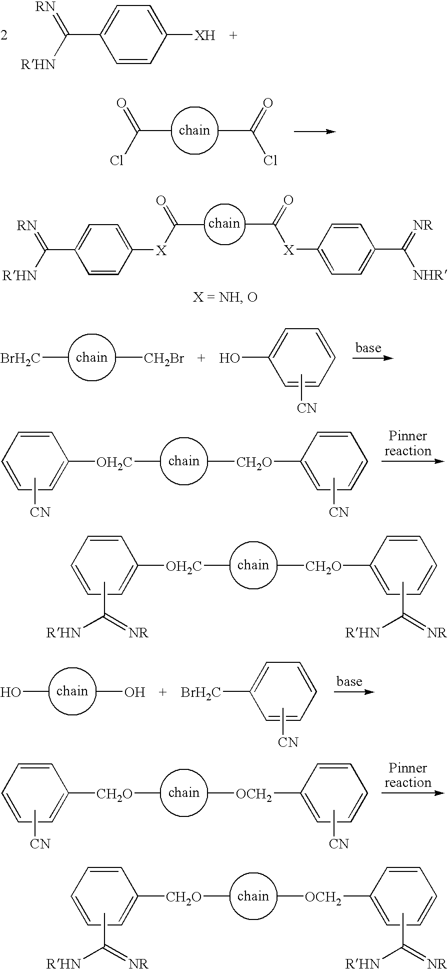

Compounds in which the (de)activating groups are amide or ester groups are obtained from a diacyl chloride and an aminobenzamidine, following classical procedures. Compounds in which the (de)activating groups are methyleneoxy or oxymethylene groups are obtained by a Williamson reaction followed by a Pinner reaction.

### General procedures for preparation: 4,4′-(1,4-piperazinediyl)bisbenzenecarboximidamides

A mixture of 4,4′-(1,4-piperazinediyl)bisbenzonitrile (2 mmol; 0.6 g) in dichloromethane (250 ml) and methanol (10 ml) was saturated with HCl gas, and the reaction medium was left at room temperature for 4 days. The precipitate (crude imidate) was filtered, washed with acetone, and treated with the appropriate amine (20 mmol) in refluxing ethanol (50 ml) for 1 h. After it cooled, the precipitate was filtered and thoroughly washed. When no precipitation occurred, the solution was concentrated under reduced pressure, and the residue was triturated with ether; the solid was filtered and thoroughly washed. Pure analytical samples were obtained without further purification.

A list of examples, which is not exhaustive, is given below:

### EXAMPLE 1

### N,N′-Dipropyl 4,4′-(1,4-piperazinediyl)bisbenzenecarboxidamide, dihydrochloride salt, (TH63N; Formula 1)

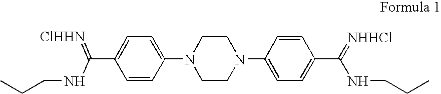

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with propylamine (20 mmol; 1.7 mL) in ethanol (50 mL) at room temperature for 3 days. The reaction medium was concentrated under reduced pressure and the residue was washed ether.

Yield: 30%.

M.p.: >300° C.

NMR (DMSO d6): 9.6-8.6 (br, 6 H); 7.7 (d, 4 H, J=9 Hz); 7.1 (d, 4 H, J=9 Hz); 3.5 (s, 8 H); 3.4 (t, 4 H); 1.6 (sext, 4 H, J=7 Hz); 0.9 (t, 6 H, J=7 Hz) ppm.

IR: 3048; 1672; 1606; 1515; 1385 cm⁻¹.

C₂₄H₃₄N₆.2HCl (479.49). Calc.: C, 60.12; H, 7.57; N, 17.53. Found: C, 59.94; H, 7.32; N, 17.61.

### EXAMPLE 2

### N,N′-Diisopropyl 4,4′-(1,4-piperazinediyl)bisbenzenecarboxidamide, dihydrochloride salt (TH141; Formula 2)

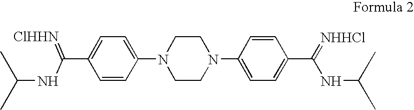

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with isopropylamine (50 mmol; 4.3 mL) in boiling ethanol (50 mL) for 90 minutes. The solution was cooled down to room temperature and concentrated under reduced pressure. The residue was washed successively with dimethylformamide and ether.

Yield: 40%

M.p.: >300° C.

NMR (DMSO d6): 9.1-8.8 (br, 6 H); 7.7 (d, 4 H, J=9 Hz); 7.1 (d, 4 H, J=9 Hz); 4.0 (m, 2 H); 3.5 (s, 8 H); 1.2 (d, 12 H, J=7 Hz) ppm.

IR: 3077; 1672; 1602; 1516, 1232 cm⁻¹

C₂₄H₃₄N₆.2HCl.0.6H₂O (490.30). Calc.: C, 59.35; H, 7.36; N, 18.05. Found: C, 58.79; H, 7.37; N, 17.54.

### EXAMPLE 3

### N,N′-Dicyclopropyl 4,4′-(1,4-piperazinediyl)bisbenzenecarboxidamide, dihydrochloride salt (TH63C; Formula 3)

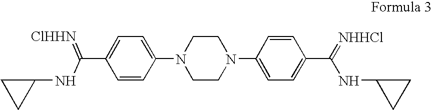

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with cyclopropylamine (25 mmol; 2.1 mL) in ethanol (50 mL) at room temperature for 3 days. The reaction medium was concentrated under reduced pressure and the residue was washed ether.

Yield: 30%

M.p.: >300° C.

NMR (DMSO d6): 9.7-8.8 (br, 6 H); 7.7 (d, 4 H, J=9 Hz); 7.1 (d, 4 H, J=9 Hz); 3.5 (s, 8 H); 2.7 (m, 2 H); 1.0-0.8 (2 m, 8 H) ppm.

IR: 3072; 1673; 1605; 1516, 1350; 1235 cm⁻¹

C₂₄H₃₀N₆.2HCl (475.47). Calc.: C, 60.63; H, 6.78; N, 17.68. Found: C, 60.51; H, 6.63; N, 17.49.

### EXAMPLE 4

### N,N′-Dicyclopentyl 4,4′-(1,4-piperazinediyl)bisbenzenecarboxidamide, dihydrochloride salt (TH137; Formula 4)

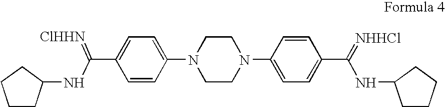

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with cyclopentylamine (40 mmol; 4.0 mL) in boiling ethanol (50 mL) for 90 minutes. The solution was cooled down to room temperature and concentrated under reduced pressure. The residue was washed successively with dimethylformamide and ether.

Yield: 30%.

M.p.: >300° C.

NMR (DMSO d6): 9.2-8.7 (br, 6 H); 7.6 (d, 4 H, J=9 Hz); 7.1 (d, 4 H, J=9 Hz); 4.1 (quint, 2 H, J=7 Hz); 3.5 (s, 8 H); 2.0 (m, 4 H, J=7 Hz); 1.7-1.5 (3 sets of multiplets, 3×4 H) ppm.

IR: 3062; 1668; 1601; 1515; 1395; 1232 cm⁻¹

C₂₈H₃₈N₆.2HCl (531.58). Calc.: C, 63.27; H, 7.58; N, 15.81. Found:. C, 59.94; H, 7.32; N, 17.61.

### EXAMPLE 5

### N,N′-Dicyclohexyl 4,4′-(1,4-piperazinediyl)bisbenzenecarboxidamide, dihydrochloride salt (TH110; Formula 5)

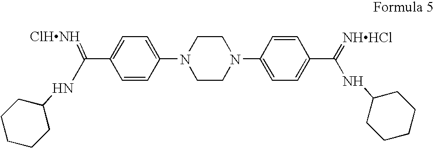

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with cyclohexylamine (20 mmol; 2.3 mL) in boiling ethanol (50 mL) for 30 minutes. The solid was filtered and successively washed with water, ethanol, and ether.

Yield: 25%.

M.p.: >300° C.

NMR (DMSO d6): 9.2 (br, 4 H); 9.1 (br, 2 H); 7.7 (d, 4 H, J=9 Hz); 7.1 (d, 4 H, J=9 Hz); 3.5 (m, 2 H); 3.5 (s, 8 H); 1.9 (d, 4 H, J=8 Hz); 1.8 (d, 4 H, J=8 Hz); 1.6 (d, 2 H, J=12 Hz); 1.4 (m, 8 H); 1.1 (m, 2 H) ppm.

IR: 3053; 1673; 1606; 1516; 1234 cm⁻¹.

C₃₀H₄₂N₆.2HCl (559.62). Calc.: C, 64.39; H, 7.92; N, 15.02. Found: C, 64.46; H, 7.72; N, 15.15.

### EXAMPLE 6

### N,N′-Dihydroxy 4,4′-(1,4-piperazinediyl)bisbenzenecarboxidamide (TH 125; Formula 6)

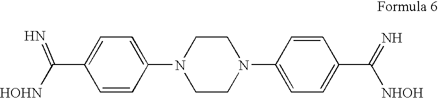

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with a boiling solution of hydroxylamine in ethanol [prepared from hydroxylamine hydrochloride (10 mmol; 0.7 g) sodium methoxide (25% in methanol, 11 mmol, 2.5 mL) and ethanol (25 mL)] for 60 minutes. The hot mixture was filtered and the precipitate was successively washed with water, ethanol, dimethylformamide, and ethanol.

Yield: 25%.

M.p.: >300 C.

NMR (DMSO d₆): 9.4 (s, 2 H); 7.5 (d, 4 H, J=9 Hz); 7.0 (d, 4 H, J=9 Hz); 5.7 (s, 4 H); 3.4 (s, 8 H) ppm.

IR: 3494; 3365; 2840; 1666; 1608; 1523; 1401; 1240 cm⁻¹.

C₁₈H₂₂N₆O₂ (354.41). Calc.: C, 61.00; H, 6.26; N, 23.71. Found: C, 61.20; H, 6.41; N, 23.39.

### EXAMPLE 7

### 4,4′-(1,4-Piperazinediyl)bisbenzenecarboxhydrazide (TH117; Formula 7)

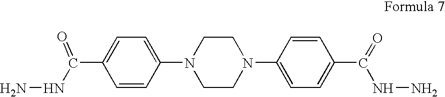

A mixture of 4,4′-(1,4-piperazinediyl)bisbenzoic acid ethyl ester (0.4 g: 1 mmol), water (0.5 ml), ethanol (2.5 ml), and hydrazine hydrate (5 ml) was heated under reflux for 8 hours. After cooling, the precipitate was filtered and retreated under the same experimental conditions for a further 8 hours period. After cooling, the precipitate was filtered and washed with water.

Yield: 80%.

M.p.: >300° C.

NMR (DMSO d6): 9.6 (br, 2H); 7.9 (d, 4 H, J=8 Hz); 7.0 (d, 4 H, J=8 H); 4.5 (br, 4 H); 3.5 (s, 8 H) ppm.

IR: 3307; 2982; 2848; 1690; 1640; 1602; 1278; 940 cm⁻¹.

C₁₈H₂₂N₆O₂ (354.41). Calc.: C, 61.00; H, 6.26; N, 23.71. Found: C, 61.26; H, 6.38; N, 23.71.

### EXAMPLE 8

### N,N′-Dimethyl 4,4′-(1,4-piperazinediyl)bisbenzenecarboxidamide, dihydrochloride salt (TH112; Formula 8)

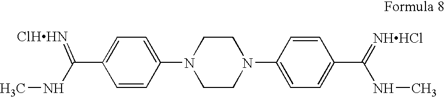

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with methylamine (50 mmol; 3.9 mL of an aqueous solution at 40%) in ethanol (50 mL) at reflux for 30 minutes. The precipitate was filtered from the hot mixture and washed with ethanol.

Yield: 40%.

M.p.: >300° C.

NMR (DMSO d6): 9.2 (br, 6 H); 7.7 (d, 4 H, J=9 Hz); 7.1 (d, 4 H, J=9 Hz); 3.6 (s, 8 H); 3.0 (s, 6 H) ppm.

IR: 3124; 2847; 1668; 1505; 1452; 1232 cm⁻¹.

C₂₀H₂₆N₆.2HCl (423.38). Calc.: C, 56.74; H, 6.67; N, 19.85. Found: C, 56.82; H, 6.53; N, 20.02.

### EXAMPLE 9

### N,N′-Dibenzyl 4,4′-(1,4-piperazinediyl)bisbenzenecarboxidamide, dihydrochloride salt (TH105; Formula 9)

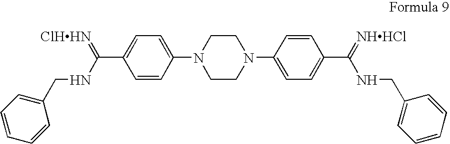

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with benzylamine (20 mmol; 2.2 mL) in ethanol (50 mL) at reflux for 30 minutes. The precipitate was filtered from the hot mixture and washed with water, ethanol, and ether.

Yield: 50%.

M.p.: >300° C.

NMR (DMSO d6): 10.0 (s, 2 H); 9.3 (s, 2 H); 8.9 (s, 2 H); 7.8 (d, 4 H, J=8 Hz); 7.4 (m, 8 H); 7.3 (t, 2 H, J=7 Hz); 7.1 (d, 4 H, J=8 Hz); 4.7 (s, 4 H); 3.6 (s, 8 H) ppm.

IR: 3099; 1668; 1607; 1518; 1384; 1235 cm⁻¹.

C₃₂H₃₄N₆.2HCl (575.57). Calc.: C, 66.78; H, 6.30; N, 14.60. Found: C, 66.9; H, 6.50; N, 14.41.

### EXAMPLE 10

### 1,4-Bis[4-(1,4,5,6-tetrahydropyrimidin-2-yl)phenyl]piperazine, dihydrochloride salt (TH142; Formula 10)

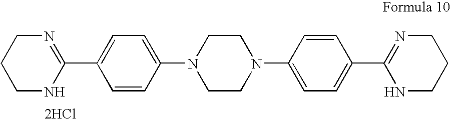

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with 1,3-diaminopropane (30 mmol; 2.5 mL) in ethanol (50 mL) at reflux for 90 minutes. After cooling, the precipitate was filtered and washed with dimethylformamide (briefly) and ether.

Yield: 25%.

M.p.: >300° C.

NMR (DMSO d6): 9.5 (br, 4 H); 7.6 (d, 4 H, J=9 Hz); 7.1 (d, 4 H, J=9 Hz); 3.5 (s, 8 H); 3.4 (t, 8 H, J=5 Hz); 1.9 (mult, 4 H, J=5 Hz) ppm.

IR: 3271, 1638, 1602, 1516, 1231 cm⁻¹.

C₂₄H₃₀N₆.2HCl (475.46). Calc.: C, 60.63; H, 6.78; N, 17.68. Found: C, ; H, ; N,

### EXAMPLE 11

### 1,4-bis[4-(1H-benzymidazol-2-yl)phenyl]piperazine, dihydrochloride salt (TH 115; Formula 11)

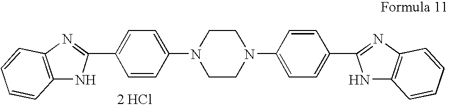

A solution of 4,4′-(1,4-piperazinediyl)bisbenzaldehyde (10 mmol; 2.94 g) in dichloromethane (100 mL) was added dropwise to a mixture of thionyl chloride (24 mmol; 1.8 mL) and pyridine (24 mmol; 2 mL) in dichloromethane (100 mL). The reaction medium was stirred for 2 hours at room temperature. 1,2-Phenylenediamine (60 mmol; 6.5 g) was slowly added and the mixture was stirred overnight. The precipitate was filtered and washed successively with water, ethanol, and dichloromethane.

Yield: 85%.

M.p.: >300° C.

NMR (DMSO d₆): 8.1 (d, 4 H, J=9 Hz); 7.6 (dd, 4 H, J=6 Hz and 3 Hz); 7.3 (dd, 4 H, J=6 Hz and 3 Hz); 7.2 (d, 4 H, J=9 Hz); 3.7 (s, 8 H) ppm.

IR: .3600-2200 (br); 1603; 1508; 1229; 1034; 745 cm⁻¹

C₃₀H₂₆N₆.2HCl (543.49). Calc.: C, 66.30; H, 5.19; N, 15.46. Found: C, 66.40; H, 5.02; N, 15.87.

### EXAMPLE 12

### N,N′-Dibutyl 4,4′-(1,4-piperazinediyl)bisbenzenecarboxidamide, dihydrochloride salt (TH 104; Formula 12)

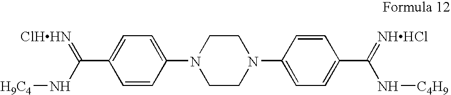

A mixture of 4,4′-(1,4-piperazinediyl)bisbenzonitrile (2 mmol; 0.6 g) in dichloromethane (250 mL) and methanol (10 mL) was treated with gaseous hydrochloric acid (10 g) and the reaction medium was left at room temperature for 4 days. The precipitate was filtered and washed with acetone. The crude imidate was used without further purification and treated with butylamine (20 mmol; 2.0 mL) in boiling ethanol (50 mL) for 30 minutes. The solid was filtered from the hot mixture and successively washed with water, ethanol, and ether.

Yield: 25%.

M.p.: >300° C.

NMR (DMSO d6): 9.2 (br, 6 H); 7.7 (d, 4 H, J=9 Hz); 7.1 (d, 4 H, J=9 Hz); 3.5 (s, 8 H); 3.4 (t, 4 H, J=8 Hz); 1.6 (quint, 4 H, J=8 Hz); 1.4 (sext, 4 H); 0.9 (t, 6 H, J=8 Hz) ppm.

IR: 3065; 1671; 1614; 1518; 1383; 1231 cm⁻¹.

C₂₆H₃₈N₆.2HCl (507.54). Calc.: C, 61.53; H, 7.94; N, 16.56. Found: C, 61.38; H, 7.71; N, 16.60.

### EXAMPLE 13

### N,N′-bis[4-(aminoiminomethyl)phenyl]pentane-1,5-dicarboxamide (TH 701; Formula 13)

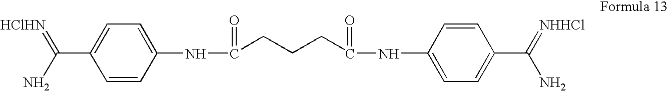

A mixture of 4-aminobenzamidine monohydrochloride (0.9 g; 5 mmol), pyridine (4 mL) and glutaryl dichloride (0.3 mL; 2.5 mmol) in dimethylformamide (20 mL) was heated under reflux for 30 minutes. After cooling, the solid was filtered and washed with acetone.

Yield: 50%.

M.p.: >300° C.

NMR (DMSO d6): 10.5 (s, 2 H); 9.1 (br, 8 H); 7.8 (s app, 8 H); 2.5 (t, 4 H, J=8 Hz); 1.9 (quint, 2 H, J=8 Hz) ppm.

IR: 3400-2300; 3094; 1923; 1659; 1604; 1351 cm⁻¹.

C₁₉H₂₂N₆O₂.2HCl (439.34). Calc.: C, 51.94; H, 5.51; N, 19.13. Found: C, 51.79; H, 5.31; N, 18.89.

### EXAMPLE 14

### N,N′-bis[4-(aminoiminomethyl)phenyl]hexane-1,6-dicarboxamide, dihydrochloride sal (TH 702; Formula 14)

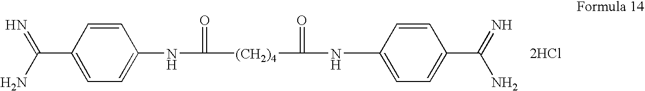

TH 702 was obtained by heating under reflux for 1 h a mixture of 4-aminobenzamidine monohydrochloride (0.86 g, 5 mmol), adipoyl dichloride (0.37 mL, 2.5 mmol), and pyridine (4 mL, 50 mmol) in N,N-dimethyl formamide (20 mL). The precipitate was filtered and thoroughly washed successively with water and acetone.

Yield: 40%.

M.p.: 294-295° C. (decomp.).

¹H NMR (DMSO d6): 10.6 (s, 2 H); 9.2 (s, 4 H); 9.0 (s, 4 H); 7.8 (dd, 8 H); 2.4 (m br, 4 H); 1.7 (m br, 4 H) ppm.

IR: 3096; 1671; 1611; 1523; 1483; 1256 cm⁻¹.

Anal. Calc. for C₂₀H₂₄N₆O_{2.}2HCl (452.15): C, 52.98; H, 5.78; N, 18.54. Found: C, 52.91; H, 5.65; N, 18.31.

### EXAMPLE 15

### N,N′-Dihydroxy 4,4′-(1,4-Piperazinediyl)bisbenzenecarboxidamide, dihydrochloride salt (TH125.HCl; Formula 15).

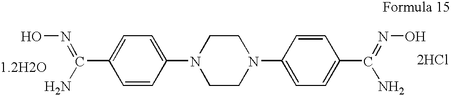

A suspension of N,N′-bis(4-cyanophenyl)piperazine (5.0 g, 17 mmol) in dimethyl formamide (DMF) (100 mL) was heated to 110° C. while stirring when a cloudy solution resulted. Hydroxylamine 50 W % solution in water (11.7 mL, 174 mmol) was diluted with DMF (11.7 mL) was added drop wise in to the above stirring reaction mixture during 12 minutes. After the addition was complete the reaction mixture was heated at 100° C. for 2 hrs until the nitrile peak at 2214 cm−1 in the IR spectrum of the reaction mixture is no longer detected. The reaction mixture was cooled to room temperature and poured on to crushed ice about 1 kg and mixed gently for 5 minutes and the ice allowed to melt down at room temperature. The resulting snow white solid was filtered, washed thoroughly with water followed by ethanol (100 mL), acetone (100 mL), hexane (50 mL) and dried under vacuum at room temperature. The crude bisamidoxime free base thus obtained was crystallized from dimethylformamide-acetone 7:1 v/v as cream white feathery needles. The product was filtered under vacuum, washed with water, ethanol, acetone (100 mL) each followed by hexane (50 mL) and dried under vacuum (5.6 g) 90% yield. Proton NMR (DMSO-d6): δ9.36 (s, 2H), 7.56 (d, J=10 Hz, 4H), 6.99 (d, J=10 Hz, 4H), 5.65 (s, 4H), 3.32 (s, 8H).

To a suspension of N,N′-Dihydroxy 4,4′-(1,4-Piperazinediyl)bisbenzenecarboxidamide (1.0 g, 2.8 mmol) in 25 mL of anhydrous DMSO was added 10% methanolic hydrochloric acid (5.0 mL, 14 mmol) and stirred magnetically at room temperature for half an hour when a clear solution resulted. The stirring continued at room temperature for an additional 3.5 hours. The resulting solution was filtered (to remove trace level particulate matter) directly in to 300 mL of anhydrous methylene chloride and gently swirled by hand for 2 minutes when a snow white precipitate resulted. It was allowed to stand at room temperature for 45 minutes and then filtered under vacuum. The product was washed thoroughly with anhydrous methylene chloride followed by of hexane (50 mL) and dried under high vacuum at room temperature for 24 hrs, 1.1 g, 91% yield.

m.p. >265° C.

Anal. (C18H25N6O3C12-2.0HCl-1.2H2O), calculated % C 48.29, % H 5.67, % N 18.77. Found % C 48.07, % H 5.73, % N 18.53.

¹H NMR (DMSO-d6): δ12.66 (s, 2H), 11.05 (Br s, 2H), 9.12 (s, 2H), 8.65 (s, 2H), 7.7 (d, J=8.5 Hz, 2H), 7.12 (d, J=8.5 Hz, 2H), 3.53 (s, 8H).

### EXAMPLE 16

### N,N′-Diacetoxy 4,4′-(1,4-Piperazinediyl)bisbenzenecarboxidamide (TH734; Formula 16)

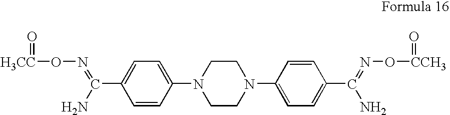

To a suspension of N,N′-Dihydroxy 4,4′-(1,4-Piperazinediyl)bisbenzenecarboxidamide (2.0 g, 5.6 mmol) in DMSO (100 mL) was added triethyl amine (1.63 mL, 11.7 mmol) and acetic anhydride (1.1 ml, 12 mmol) at room temperature. The contents were stirred overnight at room temperature for 17 hrs. An aliquot of the reaction mixture was placed in 5 mL water and the solid separated was isolated and IR spectrum was recored. The formation of a strong peak at 1742 cm⁻¹ suggests an acetate ester carbonyl absorption. The reaction mixture was poured on to crushed ice about 1.0 Kg and mixed gently. When the ice melted the separated solid was filtered under vacuum, washed thoroughly with water, ethanol followed by hexane and dried at room temperature under vacuum. The crude product was dissolved in DMSO (100 mL) and methylenechloride (50 mL) and treated with activated carbon and filtered through a bed of celite. To the filtrate was added methylenechloride (200 mL), warmed and then cooled when a cream white solid separated. The product was filtered, washed with methylenechloride (100 mL) and hexane (50 mL) and dried under vacuum (1.8 g), 73% yield. m.p. 237-239° C. Anal. (C22H26N6O4), calculated % C60.26, % H 5.98, % N 19.17. Found % C 60.56, % H 6.10, % N 18.89.

Proton NMR (DMSO-d6): δ 7.62(d, J=9.0 Hz, 4H, 7.03(d, J=9.0 Hz, 4H, 3.38(s,8H), 2.11(s,6H).

### EXAMPLE 17

### 1,4-Bis[4-(5-Methyl-1,2,4-oxadiazol-3yl)phenyl]Piperazine (TH735; Formula 17)

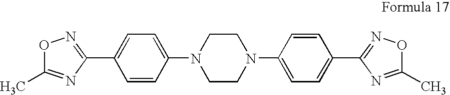

To a suspension of N,N′-Dihydroxy 4,4′-(1,4-Piperazinediyl)bisbenzenecarboxidamide (2.0 g, 5.64 mmol) in DMSO (50 mL) was added acetic anhydride (2.67 mL, 28.22 mmol) at room temperature. The contents were stirred at 80-90° C. and within half an hour a clear solution resulted. The reaction mixture was stirred at 80-90° C. for a total of 6 hrs and allowed to cool down to room temperature. The reaction flask was cooled in a freezer until the entire reaction mixture was frozen and then brought out and allowed to melt during which some residue separated. The product was quickly filtered while it was still cold and washed with hexane-ethyl acetate (2:1 v/v, 100 mL) to isolate the crude. It was crystallized from a mixture of DMF (300 mL) and acetone (500 mL) as brownish white solid. The product was filtered under vacuum, washed with methylenechloride (100 mL) followed by hexane (50 mL) and dried under vacuum, 1.4 g, 60% yield.

Anal. (C22H22N6O2), calculated % C 65.66, % H 5.51, % N 20.88. Found % C 65.87, % H 5.51, % N 20.72.

Proton NMR (DMSO-d6): δ 7.86 (d, J=9.0 Hz, 4H), 7.14 (d, J=9.0 Hz, 4H), 3.47 (s, 8H), 2.63 (s, 6H).

### EXAMPLE 18

### N,N′-Bis(4-cyanophenyl)pentanediamide (TH703; Formula 18).

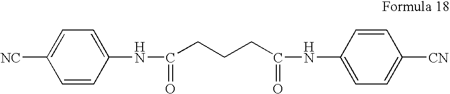

To a solution of 4-aminobenzonitrile (18.4 g, 156 mmol) in dimethyl formamide (100 mL) at room temperature was dropwise added glutaryl chloride (25 g,18.9 mL, 148 mmol) diluted with dimethyl formamide (25 mL) during 25 minutes. After the addition was completed the temperature of the reaction mixture was noticed to be at 55° C. The contents were continued stirring at room temperature for 24 hrs when a brownish white cake resulted. It was mixed with a spatula and poured on to ice-water about 1 Kg and mixed for 5 minutes. After the ice melted the product was filtered, washed thoroughly with water, ethanol (100 mL), acetone (100 mL) and finally with hexane (50 mL) and dried under vacuum 30 g, 61% yield. The product was found identical with an authentic sample (IR) earlier made at our Laboratory and homogeneous on TLC(Plastic back silica gel plate, mobile phase 100% ethyl acetate, Rf value 0.58).

### EXAMPLE 19

### N,N′-bis[4-(N-Hydroxyamidino)phenyl]pentanediamide (704; Formula 19)

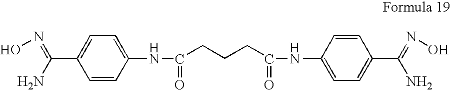

N,N′-Bis(4-cyanophenyl)pentanediamide (13 g, 39.1 mmol) was stirred in dimethyl sulfoxide (200 mL) at 70° C. for 20 minutes when a solution resulted. Hydroxylamine 50% solution in water ( 25.84 g, 24 mL, 391 mmol) diluted with dimethyl sulfoxide (24 mL) was added in to the above stirring solution at 70° C. during 15 minutes. The contents stirred overnight at 70° C. for 17 hrs when the nitrile peak (˜2214 cm−1) is no longer detected in the IR The reaction mixture was cooled to room temperature and poured on to crushed ice about 1 Kg and mixed with a spatula for 5 minutes. After the ice melted the product separated was filtered, washed thoroughly with water until the washings are neutral to pH.

The product was further washed in succession with ethanol (100 mL), acetone (100 mL), hexane (50 mL) and dried under vacuum. Snow white solid 14.0 g, 90% yield which was identical with an authentic sample (IR) earlier made at our Laboratory and was found homogeneous on TLC (Plastic back silica gel plate, mobile phase dichloromethane-methanol 3:1 v/v, Rf 0.45).

### EXAMPLE 20

### N,N′-bis[4-(N-acetoxyamidino)phenyl]pentanediamide (TH724; Formula 20)

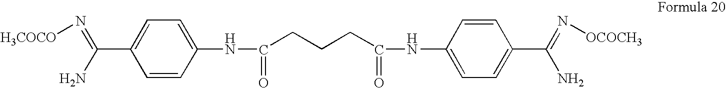

To a stirring solution of N,N′-bis[4-(N-Hydroxyamidino)phenyl]pentanediamide (1.0 g, 2.51 mmol) in dimethyl sulfoxide (25 mL) at room temperature was added triethyl amine (0.84 mL, 6 mmol) and stirred for 15 minutes. Acetic anhydride (0.472 mL, 5 mmol) was then added and the contents stirred at room temperature for 45 hrs. The reaction mixture was poured on to crushed ice about 500 g, stirred for 2 minutes and the ice allowed to melt. The white solid separated was filtered, washed thouroughly with water followed by ethanol and dried under vacuum. The product was crystallized from acetone-DMF as cream white solid 0.9 g, yield 70%, m.p. 225-228° C. Anal. C23H26N6O6, calculated % C 57.25, % H 5.43, % N 17.42. Found % C57.16, % H 5.37, % N 17.26. Proton NMR (DMSO-d6): δ 10.12 (s, 2H), 7.65-7.59 (m, 8H), 6.34 (s, 4H), 3.74(s,6H), 2.39(t, J=9.6 Hz, 4H), 2.9 (m,2H).

### EXAMPLE 21

### N,N′-Bis[4-(5-Methyl-1,2,4-oxadiazol-3yl)phenyl]pentanediamide (TH728; Formula 21)

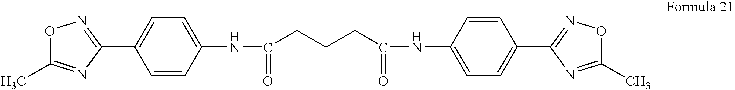

N,N′-bis[4-(N-Hydroxyamidino)phenyl]pentanediamide (2.0 g, 5.02 mmol) was stirred in DMSO (25 mL) for 15 minutes at room temperature and then acetic anhydride (2.4 mL, 25 mmol) was added and stirred for 15 minutes at room temperature when a true solution resulted. It was then heated at 80-90° C. while stirring for 24 hrs, cooled to room temperature and poured on to crushed ice (˜500 g), mixed for 2 minutes and the ice was allowed to melt. The precipitated white solid was filtered, washed thoroughly with water, ethanol (100 mL) followed by hexane (50 mL) and dried under vacuum. The product was crystallized from methylene chloride-methanol 5:1 v/v as brownish white flakes 1.6 g, yield 69%, m.p. 235-237° C. Anal. C23H22N6O4, calculated % C 61.88, % H 4.97, % N 18.83. Found % C 61.64, % H 5.18, % N 18.68. Proton NMR (DMSO-d6): δ 10.2(s, 2H), 7.93(d, J=8.5 Hz, 4H), 7.79(d, J=8.5 Hz, 4H), 2.64(s, 6H), 2.44(t, J=7.5 Hz, 4H), 1.95 (p, J=7.5 Hz, 2H).

### EXAMPLE 22

### N,N′-Bis[4-(N-propylcarbonyloxyamidino)phenyl]pentanediamide (TH740; Formula 22)

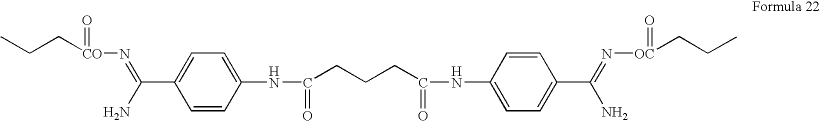

To a stirring solution of N,N′-bis[4-(N-Hydroxyamidino)phenyl]pentanediamide (3.0 g, 7.53 mmol) and triethylamine (2.51 mL, 18 mmol) in DMSO (50 mL) at room temperature was added butyric anhydride (2.45 mL, 15 mmol) at room temperature in one lot. The contents stirred at room temperature for 48 hrs and poured in to cold water (800 mL) and mixed with a glass rod for 1 minute. The white solid separated was filtered, washed thoroughly with water and dried under vacuum at room temperature. The resulting solid was crystallized from acetone-DMSO 7:1 v/v as feathery white crystals (3.0 g) yield 60%, m.p. 194-196° C. Anal. C26H34N6O6, calculated % C 59.30, % H 6.51, % N 15.96. Found: % C 59.45, % H 6.36, % N 15.83. Proton NMR (DMSO-d6): δ 10.12(s,2H), 7.65(s,8H), 6.68(s,4H),2.41(m,6H),1.94(m,2H), 1.63(m,4H),0.94(t, J=9.0 Hz).

### EXAMPLE 23

### N,N′-Bis[4-(5-Propyl-1,2,4-oxadiazol-3yl)phenyl]pentanediamide (TH741; Formula 23)

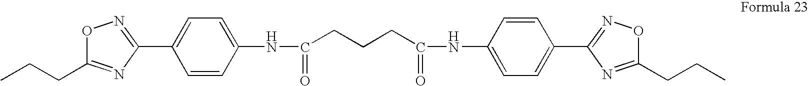

To a solution of N,N′-bis[4-(N-Hydroxyamidino)phenyl]pentanediamide (3.0 g, 7.53 mmol) in DMSO (40 mL) at room temperature was added butyric anhydride (6.2 mL, 38 mmol) and stirred for 15 minutes. The reaction mixture was stirred at 85-95° C. for 24 hrs and then cooled to room temperature. The reaction mixture was poured in to cold (10-15° C.) 2% sodium bicarbonate solution in water (800 mL) and stirred with a glass rod for 1 minute when some solid separated. The product was filtered, washed thoroughly with water and dried under vacuum at room temperature. It was crystallized from ethyl acetate-methanol 7:1 v/v as brownish white solid 2.8 g, yield 74% and m.p.196-198° C. Anal. C27H30N6O4, calculated % C 64.53, % H 6.02, % N 16.72. Found: % C 64.70, % H 6.22, % N 16.87. Found % C 64.70, % H 6.22, % N 16.87. Proton NMR (DMSO-d6): δ 10.23(s,2H), 7.94(d, J=11 Hz, 4H), 7.80(d, J=11 Hz,4H), 2.97(t, J=7.2 Hz, 4H), 2.46(t, J=7.2 Hz, 4H), 1.96(p, J=7.2 Hz, 2H), 1.81(m, J=7.2 HZ,4H), 0.98(t, J=7.2 Hz, 6H).

### EXAMPLE 24

### N,N′-Bis[4-(N-Pentylcarbonyloxyamidino)phenyl]pentanediamide (TH742; Formula 24)

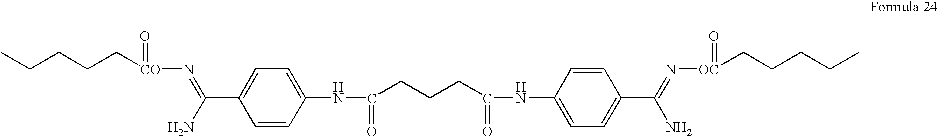

To a stirring suspension of N,N′-bis[4-(N-Hydroxyamidino)phenyl]pentanediamide (2.0 g, 5 mmol) in DMSO (25 mL) was added triethylamine (1.7 mL, 12 mmol) followed by hexanoic anhydride (2.13 g, 9.9 mmol). The contents were stirred at room temperature for 24 hrs and then poured on to crushed ice (˜800 g) and mixed gently. The white product separated was filtered after the ice melted, washed thoroughly with water and dried under vacuum at room temperature. The product was crystallized from acetone-DMSO 5:1 v/v as a shining white solid, 1.92 g, yield 64%, m.p. 182-184° C. Anal. C31H42N6O6,calculated % C 62.61, % H 7.12, % N 14.13. Found % C62.36, % H 7.01, % N 13.96. Proton NMR (DMSO-d6): δ 10.11(s,2H), 7.63(s, 8H), 6.67(s,4H), 2.43(m, 8H), 1.90(p, J=7.2 Hz,2H), 1.56(p, J=7.2, 4H), 1.27(m,8H), 0.86(t, J=7.2 Hz, 6H).

### EXAMPLE 25

### N,N′-Bis[4-(5-Pentyl-1,2,4-oxadiazol-3yl)phenyl]pentanediamide (TH743; Formula 25)

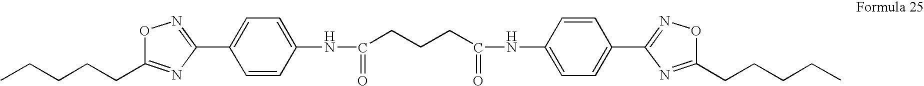

To a suspension of N,N′-bis[4-(N-Hydroxyamidino)phenyl]pentanediamide (3.0 g, 7.53 mmol) in DMSO (30 mL) was added hexanoic anhydride (8.75 mL, 38 mmol) and the contents stirred at 90-95° C. for 24 hrs. The reaction mixture was cooled to room temperature, poured in to cold 1.3% sodium bicarbonate solution in water (800 mL) and mixed for 1 minute. The resulting solid was filtered, washed thoroughly with water and dried under vacuum at room temperature.

The crude product was chromatographed on a column of silica gel (3.5×34 cm) using ethyl acetate-hexane 1:1 and 3:1 v/v as eluents. The fractions were monitored by TLC (Plastic back silica gel plate, mobile phase ethyl acetate-hexane: 19:1 v/v, Rf value of the product 0.74). The resulting product from the column was further crystallized from 100% methanol as shining white solid 2.6 g, yield 62%, m.p.171-173° C. Anal. C31H38N6O4, calculated % C 66.65, % H 6.86, % N 15.04. Found % C 66.80, % H 6.65, % N 14.92. Proton NMR (DMSO-d6): δ 10.22(s, 2H), 7.92(d, J=8.8 Hz, 4H), 7.78(d, J=8.8 Hz, 4H), 2.95(t, J=7.6 Hz, 4H), 2.44(t, J=7.2 Hz, 4H), 1.92(p, J=7.2 Hz, 2H), 1.75(m, 4H), 1.32(m, 8H), 0.85(t, J=7.2 Hz, 6H).

### EXAMPLE 26

### N,N′-Bis[4-(N-3,4-Butenecarbonyloxyamidino)phenyl]pentanediamide (TH744; Formula 26)

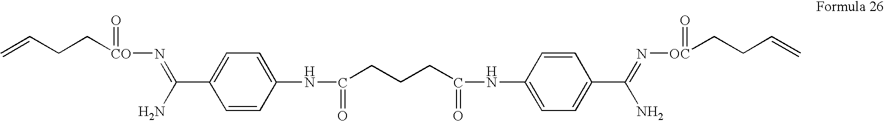

To a suspension of N,N′-bis[4-(N-Hydroxyamidino)phenyl]pentanediamide (2.0 g, 5 mmol) were added triethylamine (1.54 mL, 11 mmol) and 4-pentenoic anhydride (1.81 mL, 9.9 mmol) and stirred at room temperature for 24 hrs. The reaction mixture was poured on to crushed ice (˜800 g) and mixed gently for 1 minute with a glass rod. The snow white solid separated was filtered after the ice melted, washed thoroughly with water and dried under vacuum. The product was crystallized from acetone-DMSO 9:1 v/v as a snow white solid 2.7 g, yield 73%, m.p.178-180° C. Anal. C29H34N6O6, calculated % C 61.91, % H 6.09, % N 14.94. Found % C 61.73, % H 5.97, % N 14.88.

Proton NMR (DMSO-d6): δ 10.12(s, 2H), 7.65(s, 8H), 6.70(s,4H), 5.87(m, 2H), 5.11-4.99(m,4H), 2.55(m,4H), 2.41-2.35 (m,8H), 1.92(p, J=7.2).

### EXAMPLE 27

### N,N′-Bis[4-(5-3,4-Butene-1,2,4-oxadiazol-3yl)phenyl]pentanediamide (TH745; Formula 27)

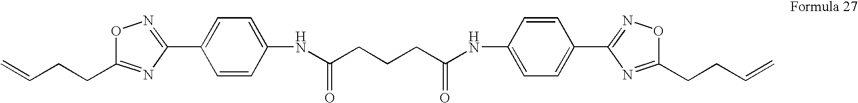

4-Pentenoic anhydride (6.42 mL, 35.12 mmol) was added to a stirring suspension of N,N′-bis[4-(N-Hydroxyamidino)phenyl]pentanediamide (2.8 g, 7 mmol) and the contents were stirred at 90-95° C. for 24 hrs. The reaction mixture was cooled to room temperature and poured in to cold (5-10° C.) 2% sodium bicarbonate and stirred for 2 minutes with a glass rod. The product separated was filtered, washed thoroughly with water and dried under vacuum at room temperature. The crude product was chromatographed on a short column (3.5×27.5 cm) of silica gel. The product was eluted with light petroleum ether: ethyl acetate (1:3 v/v) and was further crystallized from 100% acetone as cream white rosettes, 2.1 g, yield 57%, m.p. 168-170° C. Anal. C29H30N6O4, calculated % C66.15,% H 5.74, % N 15.96. Found 66.28, % H 5.69, % N 15.81.

Proton NMR (DMSO-d6): δ 10.23(s,2H), 7.94(d, J=8.8, 4H), 7.8(d, J=8.8 Hz), 5.93-5.83(m, 2H), 5.14-5.01(m, 4H), 3.09(t, J=7.6, 4H), 2.58-2.42(m,8H), 1.94(p, J=7.2, 2H).

### EXAMPLE 28

### N,N′-Bis[4-(N-Phenylcarbonyloxyamidino)phenyl]pentanediamide (TH746; Formula 28)

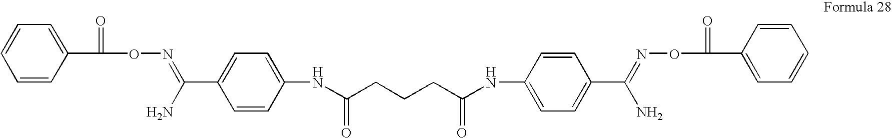

Triethylamine (0.77 mL, 5.53 mmol) was added to a stirring suspension of N,N′-bis[4-(N-Hydroxyamidino)phenyl]pentanediamide (1.0 g, 2.5 mmol) in DMSO (15 mL) followed by benzoic anhydride(0.94 mL, 5 mmol) at room temperature. The contents were stirred at room temperature for 24 hrs. Acetone 300 mL was slowly added in to the reaction mixture while stirring during 5 minutes and cooled in a freezer for 20 hrs. The snow white solid separated was filtered, washed with acetone followed by hexane 50 mL each and dried under vacuum at room temperature, 1.1 g, yield 72%, m.p. 253-255° C. Anal. C33H30N6O6, calculated % C 65.34, % H 4.98, % N 13.85. Found % C 65.50, % H 4.87, % N 13.99. Proton NMR (DMSO-d6): δ 10.15(s, 2H), 8.19(d, J=7.2 Hz, 4H), 7.74-7.64(m, 10H), 7.56-7.52(m, 4H), 6.88(s,4H), 2.44(t, J=7.2 Hz, 4H), 1.94(p, J=7.2 Hz, 2H).

### EXAMPLE 29

### N,N′-Bis[4-(5-Phenyl-1,2,4-oxadiazol-3yl)phenyl]pentanediamide (TH747; Formula 29)

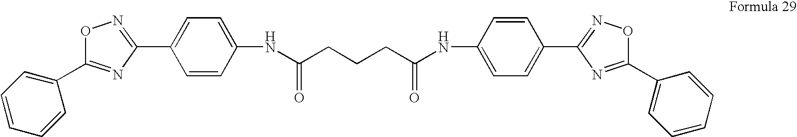

Benzoic anhydride (4.75 mL, 25 mmol) was added to a stirring suspension of N,N′-bis[4-(N-Hydroxyamidino)phenyl]pentanediamide (2.0 g, 5 mmol) in DMSO (25 mL) at room temperature and then heated at 85-95° C. for 24 hrs. The reaction mixture was cooled to room temperature. The product was stirred with 2% sodium bicarbonate (500 mL) for 30 minutes at room temperature and the solid separated was filtered, washed thoroughly with water and dried under vacuum at room temperature. The product was chromatographed on a column of silica gel (3.5×45 cm) using ethyl acetae-light petroleum ether 3:1 v/v and 100% ethyl acetate where the oxadiazole was found to elute.

The product from the column fractions was further crystallized from acetone as cream white solid, 1.75 g, yield 61%, m.p. 255-257° C. Anal. C33H26N6O4, calculated % C 69.46, % H 4.59, % N 14.73. Found % C 69.31, % H 4.40, % N 14.68.

Proton NMR (DMSO-d6): δ 10.23(s, 2H), 8.19(d, J=8.4 Hz,4H),8.05(d, J=8.4 Hz, 4H), 7.85(d, J=8.8 Hz,4H), 7.76-7.64(m,6H), 2.46(t, J=7.2 Hz, 4H), 1.96(p, J=7.2 Hz, 2H).

### EXAMPLE 30

### N,N′-Bis[4-(5H-1,2,4-oxadiazol-3yl)phenyl]pentanediamide (TH748; Formula 30)

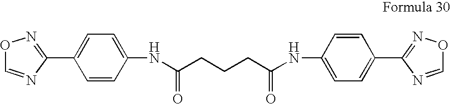

To a solution of N,N′-bis[4-(N-Hydroxyamidino)phenyl]pentanediamide (1.0 g, 2.5 mmol) in DMSO (25 mL) at room temperature was added trimethyl orthoformate (1.134 mL, 10.4 mmol) and stirred for 15 minutes. Boron trifluoride-diethyl etherate (3 drops) were added in to the reaction mixture when some white cloud of fumes were seen and disappeared in 20 minutes. The reaction mixture was allowed to stir at room temperature for 1.0 hr and then heated in an oil bath at 80-82° C. for 2 hrs. The reaction mixture was allowed to cool to room temperature and stirred with 150 mL of ethyl acetate. The ethyl acetate extract was successively washed with water, saturated sodium bicarbonate and water (50 mL) each. The ethyl acetate phase was concentrated to dryness under reduced pressure and crystallized from ethyl acetate-acetone-methanol 1:1:1 v/v as off white granules, 0.8 g and yield 76%.

Proton NMR (DMSO-d6): δ 10.30(s, 2H), 9.65(s, 2H), 7.99(d, J=8.4 Hz,4H), 7.82(d, J=8.4 Hz, 4H), 2.44(t, J=7.2 Hz, 4H), 1.94(p, J=7.2 Hz, 2H).

### EXAMPLE 31

### N,N′-Bis[4-(5-Oxo-1,2,4-oxadiazol-3yl)phenyl]pentanediamide (TH749; Formula 31).

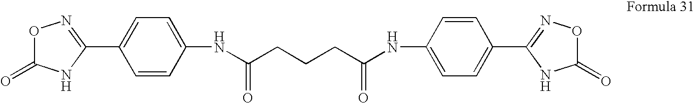

To a solution of N,N′-bis[4-(N-Hydroxyamidino)phenyl]pentanediamide (1.0 g, 2.5 mmol) in DMSO (75 mL) at room temperature was added 1,1′-carbonyldiimidazole (CDI) (1.0 g, 6.2 mmol). The contents stirred at room temperature for 30 minutes when a solution resulted. The reaction mixture was then heated at 100° C. for 2 hrs, cooled to room temperature and concentrated under high vacuum when a light brown semi-solid resulted. It was cooled in a freezer for one day when something solidified. Acetone (50 mL) was added in to the cold reaction flask, the product mixed with a spatula and filtered. The solid was washed with cold ethanol followed by cold acetone followed by hexane (50 mL) each and dried under vacuum. The product was crystallized from DMF-water as pale yellow granular solid that was filtered, washed with water (100 mL) followed by ethanol, acetone and hexane (50 m) each and dried under vacuum 0.85 g, yield 75%. Anal. C21H18N6O6, calculated % C 56.0, % H 4.03, % N 18.66.

Proton NMR (DMSO-d6): δ 10.24(s,2H), 7.78-7.70(m, 8H), 7.07(s, 2H), 2.42(t, J=7.2 Hz, 4H), 1.91(p, J=7.2 Hz, 2H).

### EXAMPLE 32

### N,N′-Bis[4(N-oxycarboxymethylamidino)phenyl]pentanediamide (TH750; Formula 32)

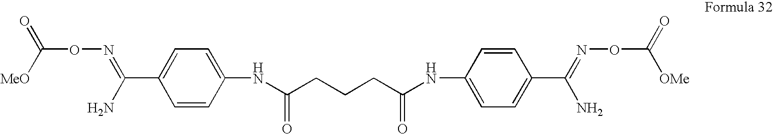

To a stirring solution of N,N′-bis[4-(N-Hydroxyamidino)phenyl]pentanediamide (0.9 g, 2.26 mmol) in dimethyl formamide (25 mL) at room temperature was added triethylamine (0.92 mL, 6.6 mmol) and the stirring continued for 10 minutes. It was then cooled in an ice bath (0-10° C.) for 10 minutes and methylchloroformate (0.5 mL, 6.5 mmol) was added in one lot while the stirring continuing. The reaction mixture was allowed to warm-up to room temperature and the stirring continued overnight for 19 hrs. A creamy brownish white suspension formed. TLC (plastic back silica gel plate, mobile phase ethyl acetate-methanol 9:1 v/v) indicated the formation of a new product with Rf value 0.55 while the standard starting material possessed the Rf value 0.23. In the reaction mixture no trace of the starting material (Rf 0.23) was found. The reaction mixture was poured on to crushed ice (˜700 g) and mixed with a glass rod for 1 minute and the snow white product filtered after the ice melted. The product was thoroughly washed with water, acetone (50 mL) followed by light petroleum ether (25 mL) and dried under vacuum at room temperature, 1.0 g, yield 86%. Anal. C23H26N6O8, calculated % C 53.69, % H 5.09, % N 16.34.

Proton NMR (DMSO-d6): δ 10.12(s,2H), 7.65-7.59(m,8H), 6.73(s, 4H), 3.74(s,6H), 2.35(t, J=7.2 Hz, 4H),1.90(p, J=7.2 Hz, 2H).

### EXAMPLE 33

### N,N′-Bis[4-(N-carboxymethylamidino)phenyl]pentanediamide (TH757; Formula 33)

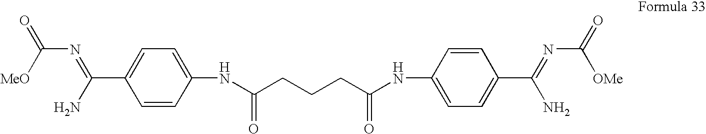

The carbamate synthesis was achieved as described below, via the Methyl 4-Nitrophenyl Carbonate route by following the general procedure of Boykin et. al. J. Med. Chem. (1999), 42, 3994-4000.

Synthesis of Methyl 4-Nitrophenyl Carbonate: To stirring solution of 4-nitrophenol (7.36 g, 53 mmol) in dichloromethane (100 mL) at 0-5° C. was added pyridine (4.4 mL, 54 mmol) and stirred for 10 minutes. Methyl chloroformate (4.1 mL, 53 mmol) diluted in dichloromethane 15 mL was added drop wise during 15 minutes and the reaction mixture allowed to warm-up to room temperature and the stirring continued for 17 hrs. The reaction mixture was diluted with dichloromethane (150 mL) and washed with 0.5N sodium hydroxide solution in water (2×100 mL) followed by water (3×200 mL). The dichloromethane solution was filtered through a bed of anhydrous sodium sulfate, concentrated to dryness and crystallized from dichloromethane-light petroleum ether as feathery white solid, 9.0 g, yield 86%.

N,N′-[4,4′(Amidinobisphenyl]pentanediamide dihydrochloride: To a suspension of 4-Aminibenzamidine dihydrochloride (10.5 g, 50.5 mmol) in DMF (200 mL) was added pyridine (41 mL, 506 mmol) and stirred for 15 minutes. Glutaryl chloride (3.2 mL, 25 mmol) diluted in DMF (15 mL) was added in to the reaction flask during 15 minutes. The contents were refluxed for 1 hr, cooled in ice-water (0-10° C.) for 30 minutes, filtered, washed with acetone (100 mL) followed by hexane (50 mL) and dried under vacuum at room temperature, 8.8 g, yield 80%.

To a suspension of N,N′-[4,4′(Amidinobisphenyl]pentanediamide dihydrochloride (0.44 g, 1.0 mmol) in DMF (20 mL) was added triethylamine (0.32 mL, 2.3 mmol) and stirred at room temperature for 30 minutes. Then methyl 4-nitrophenyl carbonate (0.45 g, 2.3 mmol) dissolved in DMF (10 mL) was added and the contents were stirred at room temperature overnight (17 hrs). The reaction mixture was poured in to ice-water (0-10° C.) and mixed with a glass rod for 1 minute. The solid separated was filtered, washed thoroughly with water and dried under vacuum at room temperature. The product was crystallized from acetone- methanol 1:1 v/v as brownish white solid 0.3 g, yield 62%.

Anal. C23H26N6O6, calculated % C 57.25, % H 5.43, % N 17.42.

Proton NMR (DMSO-d6): δ 10.20(s,2H), 9.14(Br s, 2H), 8.92(Br s, 2H), 7.94(d, J=8.4 Hz, 4H), 7.69(d, J=8.4 Hz, 4H), 3.59(s,6H),2.41(t, J=7.2 Hz, 4H), 1.90(p, J=7.2 Hz, 2H).

### EXAMPLE 34

### 1,4-Bis[4-(N-Methoxyamidino)phenyl]Piperazine (TH734; Formula 34)

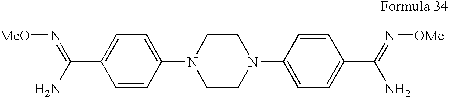

To a stirring suspension of N,N′-Dihydroxy 4,4′-(1,4-Piperazinediyl)bisbenzenecarboxidamide (2.0 g, 5.6 mmol) in DMSO (100 mL) at room temperature was added sodium methoxide powder (0.75 g, 13.9 mmol) in one lot and stirred for 0.5 hr. To the above reaction mixture was added dimethyl sulfate (1.20 mL, 12.53 mmol) in one lot at room temperature and stirred for 0.5 hr and then stirred at 85-90° C. for 20 hrs. The reaction mixture clarified and looked like honey. The reaction mixture was cooled to room temperature and poured in to water (300 mL) at 5-10° C. and stirred with a glass rod for 1 minute. The solid separated was filtered, washed thoroughly with water, ethanol followed by hexane (50 mL) each and dried under vacuum at room temperature. The solid was crystallized from DMF-acetone-water (3:2:1 v/v) as ash white solid 1.6 g, yield 74%.

Anal. C20H26N6O2, calculated % C 62.81, % H 6.85, % N 21.97.

Proton NMR (DMSO-d6): δ 7.54(d, J=8.8 Hz, 4H), 6.97(d, J=8.8 Hz, 4H), 5.87(s, 4H), 3.69 (s, 6H), 3.32 (s, 8H).

### EXAMPLE 35

### Thermal Denaturation Studies

Pentamidine, EDTA, Tris-HCl, calf thymus DNA and poly(dA-dT) used in this study were purchased from Sigma Chemical Company. The method used for the determination of the binding affinity (ΔTₘ) of the synthesized compounds to calf thymus DNA and the nucleic acid homopolymer poly(dA-dT) has been described previously (Tao et al., Eur. J. Med. Chem. 1999, 34, 1-8). The binding affinity was measured by determining the change in the midpoint (Tₘ) of the thermal denaturation curves of calf thymus DNA as well as poly(dA-dT) at a 1:5 compound to base pair ratio. Each ΔTₘ value reported in Table 1 represents the mean of at least two experimental determinations.

### EXAMPLE 36

### Pharmacology

Trypanosoma brucei brucei Lab 110 EATRO strain (pentamidine-sensitive) and clinical isolates of Trypanosoma brucei rhodesiense were used in this study. T. b. rhodesiense isolates were obtained from A. R. Njogu of the Kenya Trypanosomiasis Research Institute (KETRI: Muguga, Kenya). These included KETRI 243 (DFMO, melarsoprol, pentamidine, and berenil resistant); KETRI 243As-10-3 which is a cloned subpopulation of KETRI 243 and is refractory to arsenicals and aromatic diamidines such as berenil and pentamidine; and KETRI 269 (DFMO resistant) (Bacchi, C. J. et al., Antimicrob. Agents Chemother. 1990, 34, 1183-1188).

### EXAMPLE 37

### In Vitro Studies

The compounds were tested against trypanosome isolates grown as blood forms in HMI-18 medium containing 20% horse serum in 24 well microplates at 37° C. in 5% CO₂. Wells were inoculated with 1×10⁵ trypanosomes. The compounds were dissolved in 100% dimethylsulfoxide and diluted in the medium at the appropriate concentration so that the dimethylsulfoxide concentration did not exceed 0.3%, a non-inhibitory concentration. One-half the volume was replaced daily. Cell counts were made daily with a Coulter Counter, Model Z1 (Beckman Coulter, Miami, Fla., USA). Cells were diluted with Isoton I buffer (Beckman Coulter) and the aperture was standardized at 5.14 μm. Background checks were performed daily on 1:10 dilutions of medium. Counts, normalization, and coincident counts were accounted for by the standardized Coulter analysis program. Occasionally, hemocytometer counts were performed as a check on the validity of the Coulter program. IC₅₀ values were determined after 48 h from semi-log plots and the values are the result of duplicate determinations. Initially a broad concentration curve was used and then a close concentration curve from which IC₅₀ values were determined. Assays were done in duplicate and each point was the average of the two. Control cells grew to 5×10⁶/ml.

### EXAMPLE 38

### In Vivo Studies

Female Swiss-Webster mice (weight, 20 g) were infected (intraperitoneally) with 2.5×10⁵ trypanosomes from rat blood, and the infection was allowed to develop for 24 h before drug treatment was begun. Groups of three mice each were injected (intraperitoneally) with each drug concentration. All of the experiments included a group of untreated controls. Untreated mice died 4 to 13 days after infection, depending on the isolate. Parasitemia of animals dying of infection averaged 0.5-1.0×10⁹/mL of blood. These infections are very predictable and daily counts were not done because of the labor involved. The procedures used are standard for our laboratory. Animals were monitored weekly for parasites in tail vein blood smears. Mice were considered cured if they survived more than 30 days after the death of the last control with no parasites in tail vein blood smears. MSD (mean survival in days) was also recorded for each group. It is the average time of survival of the animals in the group, exclusive of cured animals.

IC₅₀ values were determined for pentamidine (TABLE 1) and pentamidine congeners (TABLES 2-12) tested against trypanosome isolates (T. b. brucei LAB 110 EATRO and T. b. rhodesiense KETRI 243) grown as bloodforms in HMI-18 medium containing 20% fetal bovine serum. Coulter counts were made daily and IC₅₀ values determined after 48 hours exposure. The reference compound is pentamidine.

TABLE 1
Pentamidine

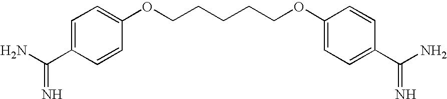

| Lab 100 | KETRI | DNA Binding |
|---|---|---|
| EATRO | 243 | Δ Tₘ (° C.) |

| | IC₅₀ | IC₅₀ | Calf | Poly |
|---|---|---|---|---|
| Compound | (μM) | (μM) | thymus | (dA-dT) |
| Pentamidine | 0.00212 | 0.00232 | 11.1 | 20.6 |

TABLE 2
Piperazine-linked bisbenzamidines, bisbenzamidoximes, and analogs

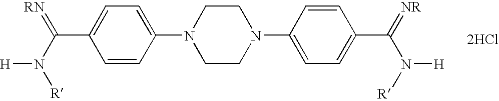

| | | | Lab 100 | KETRI | DNA Binding | |
|---|---|---|---|---|---|---|
| | | | EATRO | 243 | Δ Tₘ (° C.) | |
| | | | IC₅₀ | IC₅₀ | Calf | Poly |
| CODE | R | R′ | (μM) | (μM) | thymus | (dA-dT) |
| 0104 | H | —C₄H₉ | 0.0135 | 0.0189 | 15.2 | 23.9 |
| 0110 | H | 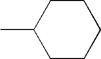 | | | 15.5 | 23.6 |
| 0105 | H | 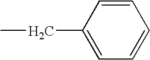 | 0.051 | 0.0525 | 18.0 | 25.1 |
| 137 | H | 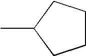 | 0.0249 | 0.0375 | 14.0 | 23.0 |
| 0103 | H | H | 0.0167 | 0.0089 | 17.0 | 23.8 |
| 0112 | H | —CH₃ | 0.205 | 0.033 | 15.2 | 22.2 |
| 63N | H | —C₃H₇ | 0.035 | 0.0325 | 17.0 | 26.0 |
| 63C | H | 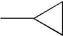 | | | 14.5 | 23.9 |
| 63I | H | 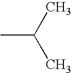 | | | 14.9 | 22.5 |
| 0125 | H | —OH | 1.025 | 1.925 | 0.8 | |
| 0140 | H | —C₂H₅ | 0.013 | 0.033 | 15.3 | 18.6 |
| 0143 | H | 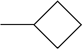 | 0.006 | 0.051 | 16.4 | — |
| 0127 | H | —C₅H₁₁ | 0.003 | | 16.9 | 22.5 |
| 0135 | H | 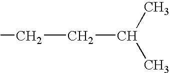 | 0.072 | 0.061 | 15.3 | 21.8 |
| 0138 | H | 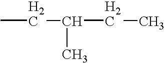 | 0.013 | 0.039 | 12.9 | 16.0 |
| 0128 | H | —C₆H₁₃ | 0.013 | 0.0239 | 10.7 | 10.1 |
| 0133 | H | —C₇H₁₅ | 0.100 | 0.057 | 8.7 | 8.5 |
| 0129 | H | 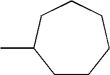 | 0.018 | 0.022 | 15.6 | 18.7 |
| 0130 | H | —C₈H₁₇ | 0.00365 | 0.019 | 4.2 | — |
| 0134 | H | 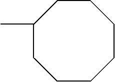 | 0.0023 | | 12.6 | 15.7 |
| 0132 | H | —C₉H₁₉ | 0.100 | 0.210 | 1.3 | 2.7 |
| 0131 | H | —C₁₀H₂₁ | 0.180 | 0.220 | 1.3 | — |
| 0136 | H | —C₁₂H₂₅ | 0.660 | 0.125 | 0.6 | 0.0 |
| 739B | H | —OCH₃ | | | | |

| | | | | | |
|---|---|---|---|---|---|
| 0126 | 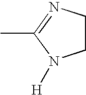 | 0.016 | 0.0065 | 15.0 | 19.4 |
| 0115 | 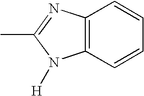 | 1.15 | 5.8 | 0.1 | 2.0 |
| 632N | 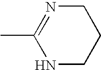 | | | 12.2 | 17.9 |

TABLE 3
1,4-Diaryl piperazine derivatives

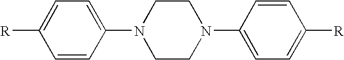

| | | Lab 100 | KETRI | DNA Binding | |
|---|---|---|---|---|---|
| | | EATRO | 243 | Δ Tₘ (° C.) | |
| | | IC₅₀ | IC₅₀ | Calf | Poly |
| CODE | R | (μM) | (μM) | thymus | (dA-dT) |
| 0117 | 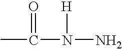 | 0.240 | | 0.1 | 0.0 |
| 0119 | —NO₂ | 0.137 | 0.250 | 0.1 | 0.0 |
| 0101 | —CN | 2.89 | 1.05 | 0.1 | −0.3 |
| 0116 | 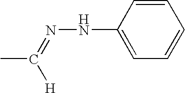 | 1.74 | 1.87 | −0.2 | 0.0 |
| 0108 | 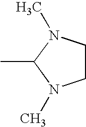 | 6.80 | 2.32 | 2.4 | 1.8 |
| 0109 | 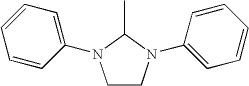 | 0.120 | 0.335 | −1.2 | 0.0 |
| 0111 | 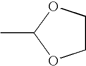 | 5.80 | 2.30 | −1.2 | 0.1 |
| 0118 | 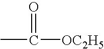 | 0.340 | 0.350 | 0.6 | 0.0 |
| 0107 | 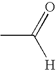 | | | −0.2 | 0.0 |
| 0123 | 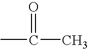 | 2.35 | 1.55 | 0.1 | 0.0 |

TABLE 4
Homopiperazine-linked bisbenzamidines and bisbenzamidoximes

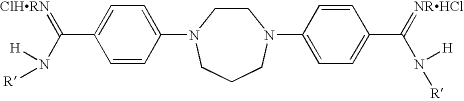

| | | | Lab 100 | KETRI | DNA Binding | |
|---|---|---|---|---|---|---|
| | | | EATRO | 243 | Δ Tₘ (° C.) | |
| | | | IC₅₀ | IC₅₀ | Calf | Poly |
| CODE | R | R′ | (μM) | (μM) | thymus | (dA-dT) |
| 74N | H | —C₄H₉ | 0.180 | 0.079 | 11.8 | 13.3 |
| 7AM | H | H | 0.00215 | 0.00270 | 15.0 | 23.1 |
| 732N | 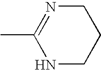 | | | | 15.5 | 21.3 |
| 73C | H | 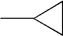 | 0.870 | 0.810 | 15.3 | 23.6 |
| 811 | H | —OH | | | | |
| 812 | H | —OCH₃ | | | | |

TABLE 5
Piperidine-linked bisbenzimidazolines

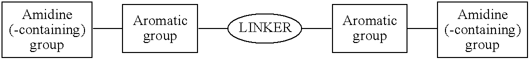

| | | | | Lab 100 | KETRI | DNA Binding | |
|---|---|---|---|---|---|---|---|
| | Amidine- | | | EATRO | 243 | Δ Tₘ (° C.) | |
| | containing | Aromatic | | IC₅₀ | IC₅₀ | Calf | Poly |
| CODE | group | group | LINKER | (μM) | (μM) | thymus | (dA-dT) |
| 4CF | 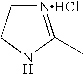 | 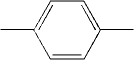 | 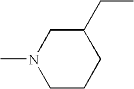 | 0.086 | 0.085 | 9.9 | 14.1 |
| 3SF | 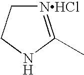 | 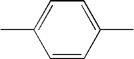 | 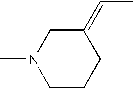 | 0.071 | 0.084 | 12.0 | 15.0 |

TABLE 6
Alkanediamide-linked bisbenzamidines,
bisbenzamidoxime, and analogs

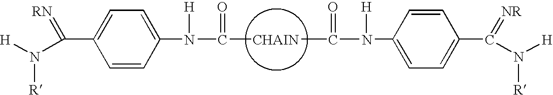

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |
| 0702 | —(CH₂)₄— | H | H | 0.0028 | 0.00245 | 8.7 | 14.4 |
| 0701 | —(CH₂)₃— | H | H | 0.0087 | 0.096 | 4.85.6 | 4.511.4 |
| 722B | —(CH₂)₃— | H | OCH₃ | | | | |
| 0704 | —(CH₂)₃— | H | OH | 7.3 | 10.0 | | |
| 0705 | —(CH₂)₆— | H | H | 0.0028 | 0.00145 | | |
| 0706 | —(CH₂)₅— | H | H | 0.00162 | 0.00205 | | |
| 0707 | —(CH₂)₂— | H | H | 9.0 | 2.19 | | |
| 0708 | —(CH₂)₇— | H | H | 0.40 | 0.24 | | |
| 0709 | —(CH₂)₈— | H | H | 0.002 | 0.0041 | | |
| 0711 | —(CH₂)₁₀— | H | H | 0.0084 | 0.0066 | | |

TABLE 7
Hexanediamide-linked bisbenzamidines and analogs

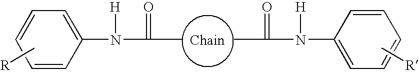

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |

| | | | | | |
|---|---|---|---|---|---|
| 0712 | —(CH₂)— | Para-CN | Para-CN | 5.0 | 1.6 |
| 0713 | nil | Para-CN | Para-CN | 0.68 | 0.34 |
| 0714 | nil | Para-H | Para-H | — | 0.155 |
| 0715 | —(CH₂)₄— | Meta- | Meta- | 0.041 | 0.021 |
| | | amidine | amidine | | |
| 0716 | —(CH₂)₄— | Para-amide | Para-amide | 2.8 | 1.1 |
| 0717 | —(CH₂)₄— | Meta- | Meta- | — | 1.975 |
| | | amide | amide | | |
| 0721 | —(CH₂)₄— | Para- | Meta- | 0.0125 | 0.007 |
| | | amidine | amidine | | |

TABLE 8
Aryl- or cycloalkyl-linked bisbenzamidines and bisbenzamidoximes

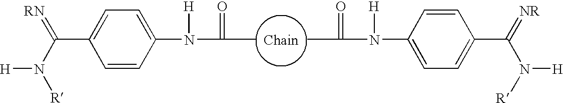

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |
| 0322 | 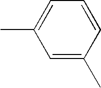 | H | H | 0.070 | 0.070 | 8.0 | 9.9 |
| 0323 | | H | OH | | — | 0.6 | 0.1 |
| 0332 | 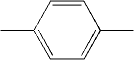 | H | H | 0.009 | 0.040 | 9.7 | 7.7 |
| 0333 | | H | OH | | — | 0.6 | 0.0 |
| 8AM | 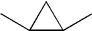 | H | H | >1.0 | >1.0 | 7.1 | 11.1 |

| | | | | | |
|---|---|---|---|---|---|
| 8IM | 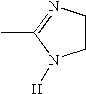 | >1.0 | 0.440 | 6.0 | 9.6 |

TABLE 9
Phenylenediamide-linked bisbenzamidoximes

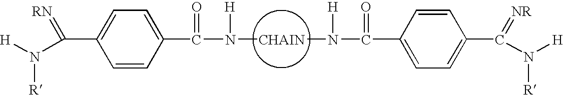

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |
| 0343 | 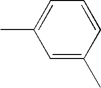 | H | OH | | | −0.3 | 0.1 |
| 0353 | 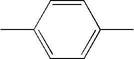 | H | OH | | | 0.4 | 0.1 |

TABLE 10
Ester derivatives

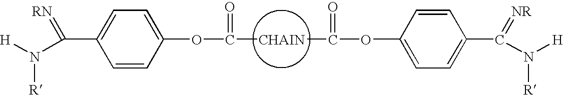

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |
| 0432 | 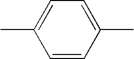 | H | H | 4.30 | 3.10 | 9.9 | — |

TABLE 11
Ether and related derivatives

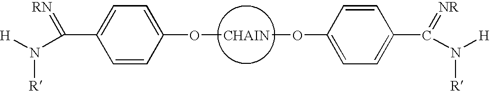

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |
| 0508 | 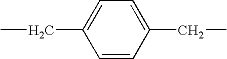 | H | H | 0.000295 | 0.000665 | 12.5 | 18.0 |
| 0513 | 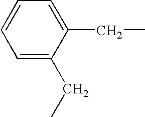 | H | H | 0.00125 | 0.00255 | −0.2 | −0.1 |
| 0512 | 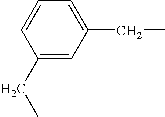 | H | H | 0.00074 | 0.0015 | 14.0 | |

TABLE 12
Phenylenedioxymethylene-linked bisbenzamidine

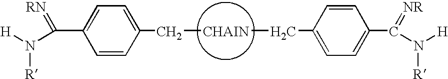

| | | | | Lab 100 | KETRI | DNA Binding | |
|---|---|---|---|---|---|---|---|
| | | | | EATRO | 243 | Δ Tₘ (° C.) | |
| | | | | IC₅₀ | IC₅₀ | Calf | Poly |
| CODE | CHAIN | R | R′ | (μM) | (μM) | thymus | (dA-dT) |
| 0519 | 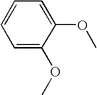 | H | H | 0.0013 | 0.0021 | 0.1 | −0.1 |

In vivo efficacy of pentamidine analogs given via i.p route vs. T. b. brucei LAB 110 EATRO is shown in TABLE 13. Mice were infected with 250,000 parasites and dosing commenced 24 h. post infection. Mice were separated into groups of three and given single i.p doses for 3 days unless otherwise noted. Infected untreated controls were used for each experiment. Mice were considered cured if surviving more than 30 days beyond death of controls without parasites in tail vein blood smears. MSD, mean survival in days, exclusive of cured animals.

TABLE 13
In vivo efficacy of pentamidine analogs given via i.p. route vs.
T. b. brucei LAB 110 EATRO, except analog 125, which was given per os

| | Dosage | MSD | # Cured/ |
|---|---|---|---|
| Compound # | (mg/kg/day) | (Days) | #Treated (%) |
| None | — | 5.0 | — |
| Pentamidine | 1, 2.5, 5, 10, 25 | >30 | 5/5* |
| 0104 | 1.0 | 7 | 0/3 |
| 0104 | 2.5 | 9 | 0/3 |
| 0104 | 5 | 18 | 5/6 (83) |
| 0104 | 10 | 6.3 | 3/6 (50) |
| 0105 | 1 | 7 | 0/3 |
| 0105 | 2.5 | 9 | 0/3 |
| 0105 | 5 | 13 | 0/3 |
| 0105 | 10 | 5 | 0/3 |
| 0105 | 15 | 6 | 0/3 |
| 0128 | 1, 2.5, 5, 10, 15, 25 | 5.3-10.6 | 0/3^{a} |
| 0129 | 1 | 7.5 | 0/3 |
| 0129 | 2.5 | 8.3 | 0/3 |
| 0129 | 5 | 9 | 4/6 (66) |
| 0129 | 7.5 | >30 | 3/3 (100) |
| 0129 | 10 | >30 | 6/6 (100) |
| 0129 | 15 | >30 | 3/3 (100) |
| 125 | 50 (po) | 5 | 0/3 |
| 125 | 100 (po) | 5 | 0/3 |
| 125 | 100 (split dose, po)^{b} | >30 | 3/3 (100) |
| 0130 | 1, 2.5, 5, 10, 15, 20 | 5 | 0/3^{a} |
| 0131 | 1, 2.5, 5, 10, 15, 25 | 5 | 0/3^{a} |
| 0137-65 C | 1 | 5 | 0/3 |
| 0137-65 C | 2.5 | 6.6 | 0/3 |
| 0137-65 C | 5 | 16 | 2/3 (66) |
| 0137-65 C | 10 | >30 | 3/3 (100) |
| 0140 | 1 | 5 | 0/3 |
| 0140 | 2.5 | 5 | 0/3 |
| 0140 | 5 | 18 | 2/3 (66) |
| 0140 | 10 | 17 | 2/3 (66) |
| 0143 | 1 | 5 | 0/3 |
| 0143 | 2.5 | 5 | 0/3 |
| 0143 | 5 | 11 | 2/3 (66) |
| 0143 | 10 | >30 | 3/3 (100) |
| 0332 | 1 | 11.5 | 0/3 |
| 0332 | 2.5 | 11.6 | 0/3 |
| 0332 | 5 | 4 | 2/3 (66) |
| 0332 | 10 | 14 | 2/3 (66) |
| 0332 | 15 | >30 | 3/3 (100) |
| 0332 | 25 | >30 | 6/6 (100) |
| 0332 | 25 (single dose) | 11.6 | 0/3 |
| 0332 | 25 (2 doses) | >30 | 3/3 (100) |
| 0508 | 1, 2.5, 5, 10, 15, 25 | 5-17.3 | 0/3^{a} |
| 0512 | 1 | 8 | 0/3 |
| 0512 | 2.5 | 17.7 | 0/3 |
| 0512 | 5 | 18 | 0/3 |
| 0512 | 10 | >30 | 3/3 (100) |
| 0512 | 15 | >30 | 3/3 (100) |
| 0513 | 1, 2.5, 5, 10, 15, 25 | 5 | 0/3^{a} |
| 0519 | 1, 2.5, 5, 10, 15, 25 | 5 | 0/3^{a} |
| 0701 | 1 | 6 | 0/3 |
| 0701 | 2.5 | 11.3 | 0/3 |
| 0701 | 5 | 10 | 2/3 (66) |
| 0701 | 10 | >30 | 3/3 (100) |
| 0701 | 15 | >30 | 3/3 (100) |
| 0701 | 25 | >30 | 3/3 (100) |
| 0701 | 15 (1x) | 14.6 | 0/3 |
| 0701 | 15 (2x) | 20 | 2/3 (66) |
| 0702 | 1 | 6.7 | 0/3 |
| 0702 | 2.5 | 10 | 0/3 |
| 0702 | 5 | >30 | 3/3 (100) |
| 0702 | 10 | >30 | 3/3 (100) |
| 0702 | 15 | >30 | 3/3 (100) |
| 0702 | 25 (1x) | 22 | 0/3 |
| 0702 | 25 (2x) | >30 | 3/3 (100) |
| 0705 | 1 | 10 | 2/3 (66) |
| 0705 | 2.5 | >30 | 3/3 (100) |
| 0705 | 5 | >30 | 3/3 (100) |
| 0705 | 10 | >30 | 3/3 (100) |
| 0705 | 25 | >30 | 3/3 (100) |
| 0706 | 1 | >30 | 3/3 (100) |
| 0706 | 2.5 | >30 | 3/3 (100) |
| 0706 | 5 | >30 | 3/3 (100) |
| 0706 | 10 | >30 | 3/3 (100) |
| 0706 | 15 | >30 | 3/3 (100) |
| 0709 | 1 | 12 | 2/3 (66) |
| 0709 | 2.5 | >30 | 3/3 (100) |
| 0709 | 5 | >30 | 3/3 (100) |
| 0709 | 10 | >30 | 3/3 (100) |
| 0709 | 25 | >30 | 3/3 (100) |
| 0711 | 1, 2.5, 5, 10, 15, 25 | 5-10.3 | 0/3^{a} |
| 0717 | 1, 2.5, 5, 10, 15, 25 | 5 | 0/3^{a} |

^{a}All doses failed to cure
^{b}Split dose = One dose given orally at 9am and the other dose given orally at 4 pm
*All doses cured

Efficacy in vivo of pentamidine analogs vs T. b. rhodesiense clinical isolates (KETRI isolates) is shown in TABLE 14. Mice were infected on day 1 with 2.5×10⁵ trypanosomes and the infections allowed to progress 24 h. prior to dosing. Animals were dosed 1×/day i.p for 3 days. Cured animals survived >30 days beyond the death of untreated, infected controls. MSD=mean survival in days exclusive of cured animals. Dose as mg/kg/day.

TABLE 14
In vivo efficacy of pentamidine analogs vs. T.b. rhodesiense
clinical isolates (KETRI isolates)

| | | | | #Cured/ |
|---|---|---|---|---|
| Isolate | Compound # | | MSD | Total |
| | | Dose* | | |
| KETRI 2002 | 0701 | — | 9 | 0/3 |
| KETRI 2002 | 0701 | 10 | >30 | 3/3 (100) |
| KETRI 2002 | 0701 | 15 | >30 | 3/3 (100) |
| KETRI 2002 | 0701 | 25 | >30 | 3/3 (100) |
| KETRI 2002 | 0702 | 10 | >30 | 3/3 (100) |
| KETRI 2002 | 0702 | 15 | >30 | 3/3 (100) |
| KETRI 2002 | 0702 | 25 | >30 | 3/3 (100) |
| KETRI 2002 | 0705 | 1 | >30 | 3/3 (100) |
| KETRI 2002 | 0705 | 2.5 | >30 | 3/3 (100) |
| KETRI 2002 | 0705 | 5 | >30 | 3/3 (100) |
| KETRI 2002 | 0705 | 10 | >30 | 3/3 (100) |
| KETRI 2002 | 0705 | 25 | >30 | 3/3 (100) |
| KETRI 2002 | 0706 | 1 | >30 | 3/3 (100) |
| KETRI 2002 | 0706 | 2.5 | >30 | 3/3 (100) |
| KETRI 2002 | 0706 | 5 | >30 | 3/3 (100) |
| KETRI 2002 | 0706 | 10 | >30 | 3/3 (100) |
| KETRI 2002 | 0706 | 15 | >30 | 3/3 (100) |
| KETRI 2002 | 0709 | 1 | 18 | 0/3 |
| KETRI 2002 | 0709 | 2.5 | >23.6 | 0/3 |
| KETRI 2002 | 0709 | 5 | 33 | 2/3 (66) |
| KETRI 2002 | 0709 | 10 | >30 | 3/3 (100) |
| KETRI 2002 | 0709 | 15 | >30 | 3/3 (100) |
| KETRI 2002 | 0709 | 25 | >30 | 3/3 (100) |
| KETRI 2002 | 0332 | 10 | >30 | 3/3 (100) |
| KETRI 2002 | 0332 | 15 | >30 | 3/3 (100) |
| KETRI 2002 | 0332 | 25 | >30 | 3/3 (100) |
| | | Dose | | |
| KETRI 2538 | — | — | 4.3 | 0/3 |
| KETRI 2538 | 0701 | 10 | >30 | 3/3 (100) |
| KETRI 2538 | 0701 | 15 | >30 | 3/3 (100) |
| KETRI 2538 | 0701 | 25 | >30 | 3/3 (100) |
| KETRI 2538 | 0702 | 5 | >30 | 3/3 (100) |
| KETRI 2538 | 0702 | 10 | >30 | 3/3 (100) |
| KETRI 2538 | 0702 | 15 | >30 | 3/3 (100) |
| KETRI 2538 | 0332 | 10 | >30 | 3/3 (100) |
| KETRI 2538 | 0332 | 15 | >30 | 3/3 (100) |
| KETRI 2538 | 0332 | 25 | >30 | 3/3 (100) |
| KETRI 1992 | — | — | 13.5 | 0/3 |
| KETRI 1992 | 0701 | 10 | 17.5 | 0/3 |
| KETRI 1992 | 0701 | 15 | 14.5 | 0/3 |
| KETRI 1992 | 0701 | 25 | 25 | 0/3 |
| KETRI 1992 | 0702 | 5 | 16.5 | 0/3 |
| KETRI 1992 | 0702 | 10 | 17.5 | 0/3 |
| KETRI 1992 | 0702 | 15 | 22 | 0/3 |
| KETRI 1992 | 0705 | 1 | 15.3 | 0/3 |
| KETRI 1992 | 0705 | 2.5 | 17.3 | 0/3 |
| KETRI 1992 | 0705 | 5 | 22.6 | 0/3 |
| KETRI 1992 | 0705 | 10 | 29.6 | 0/3 |
| KETRI 1992 | 0706 | 1 | 14 | 0/3 |
| KETRI 1992 | 0706 | 2.5 | 15.3 | 0/3 |
| KETRI 1992 | 0706 | 5 | 18.3 | 0/3 |
| KETRI 1992 | 0706 | 10 | 18.6 | 0/3 |
| KETRI 1992 | 0706 | 25 | 19.3 | 0/3 |
| KETRI 1992 | 0709 | 1 | 12.6 | 0/3 |
| KETRI 1992 | 0709 | 2.5 | 13.0 | 0/3 |
| KETRI 1992 | 0709 | 5 | 13.7 | 0/3 |
| KETRI 1992 | 0709 | 10 | 15.7 | 0/3 |
| KETRI 1992 | 0709 | 25 | 26.6 | 0/3 |
| KETRI 1992 | 0332 | 10 | 14 | 0/3 |
| KETRI 1992 | 0332 | 15 | 16.5 | 0/3 |
| KETRI 1992 | 0332 | 25 | 25 | 0/3 |

^{a}All doses failed to cure
*All doses cured

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

The invention claimed is:

1. A method of treating infection caused by a parasitic hemoflagellate, comprising administering to a mammal in need thereof an effective amount of a compound having the following structure: 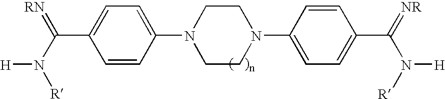
wherein R is selected from the group consisting of H and n-alkyl; wherein R′ is selected (independently from R) from the group consisting of —H, —OH, —OCH₃, n-alkyl, branched alkyl, and cycloalkyl; and wherein n is an integer value from 1 to 2.

2. The method of claim 1 wherein the infection caused by a parasitic hemoflagellate is African trypanosomiasis.

3. The method of claim 2, wherein the African trypanosomiasis is caused by Trypanosoma brucei gambiense.

4. The method of claim 2, wherein the African trypanosomiasis is caused by Trypanosoma brucei rhodesiense.

5. The method of claim 2, wherein the n-alkyl or branched alkyl chain is selected from the group consisting of ethyl, propyl, isopropyl, butyl, 2-methylbutyl, pentyl, isopentyl, hexyl, heptyl, and octyl.

6. The method of claim 2, wherein the cycloalkyl is selected from the group consisting of cyclobutyl, cyclopentyl, cycloheptyl, and cyclooctyl.

7. A method of treating infection caused by a parasitic hemoflagellate, comprising administering to a mammal in need thereof an effective amount of a compound having the following structure: 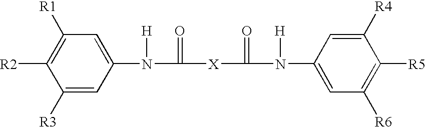
wherein X is an aromatic C₆H₄ ring or (CH₂)ₙ; wherein n is an integer value from 1 to 10; wherein R1, R2, R3, R4, R5, and R6 are, independently, —H, or —C(N═H)NHR′; wherein R′ is selected from the group consisting of —H, —OH, —OCH₃, n-alkyl, branched alkyl, and cycloalkyl.

8. The method of claim 7, wherein X is (CH₂)ₙ; wherein n is an integer value from 1 to 10; wherein R1, R3, R4, and R6 are —H; and wherein R2 and R5 are —C(N═H)NH₂.

9. The method of claim 7, wherein X is (CH₂)ₙ; wherein n is an integer value from 1 to 10; wherein R1, R2, R4, and R5 are —H; and wherein R3 and R6 are —C(N═H)NH₂.

10. The method of claim 7, wherein X is (CH₂)ₙ; wherein n is an integer value from 1 to 10; wherein R1, R3, R4, and R5 are —H; and wherein R2 and R6 are —C(N═H)NH₂.

11. The method of claim 7, wherein X is an aromatic C₆H₄ ring; wherein R1, R3, R4, and R6 are —H; and wherein R2 and R5 are —C(N═H)NH₂.

12. A bis-benzamidine of the following structure: 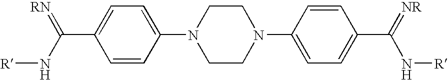
wherein R is selected from the group consisting of H and n-alkyl; and wherein R′ is selected (independently from R) from the group consisting of —H, —OH, —OCH₃, n-alkyl, branched alkyl, and cycloalkyl.

13. The compound according to claim 12, wherein the n-alkyl or branched alkyl chain is selected from the group consisting of ethyl, propyl, isopropyl, butyl, 2-methylbutyl, pentyl, isopentyl, hexyl, heptyl, and octyl.

14. The compound according to claim 12, wherein the cycloalkyl is selected from the group consisting of cyclobutyl, cyclopentyl, cycloheptyl, and cyclooctyl.

15. A bis-benzamidine of the following structure: 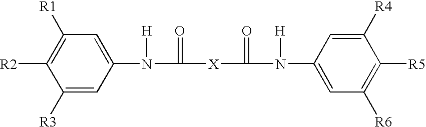
wherein X is an aromatic C₆H₄ ring or (CH₂)ₙ; wherein n is an integer value from 1 to 10; wherein R1, R2, R3, R4, R5, and R6 are, independently, —H, or —C(N═H)NHR′; wherein R′ is selected from the group consisting of —H, —OH, —OCH₃, n-alkyl, branched alkyl, and cycloalkyl.

16. The compound according to claim 15, wherein X is (CH₂)ₙ; wherein n is an integer value from 1 to 10; wherein R1, R3, R4, and R6 are —H; and wherein R2 and R5 are —C(N═H)NH₂.

17. The compound according to claim 15, wherein X is (CH₂)ₙ; wherein n is an integer value from 1 to 10; wherein R1, R2, R4, and R5 are —H; and wherein R3 and R6 are —C(N═H)NH₂.

18. The compound according to claim 15, wherein X is (CH₂)ₙ; wherein n is an integer value from 1 to 10; wherein R1, R3, R4, and R5 are —H; and wherein R2 and R6 are —C(N═H)NH₂.

19. The compound according to claim 15, wherein X is an aromatic C₆H₄ ring; wherein R1, R3, R4, and R6 are —H; and wherein R2 and R5 are —C(N═H)NH₂.
